(19) United States
(12) Patent Application Publication
(10) Pub. No.: US 2026/0345621 A1
(43) Pub. Date: Nov. 5, 2026

(54) GABAA RECEPTOR MODULATORS AND USES THEREOF

(71) Applicant: Engrail Therapeutics, Inc.

(72) Inventors: Jordi Serrats, San Diego, CA (US); Krishna Vadodaria, San Diego, CA (US); Kimberly Vanover, San Diego, CA (US); Eve Taylor, San Diego, CA (US); Stephen Cunningham, San Diego, CA (US)

(21) Appl. No.: 19/118,255

(22) PCT Filed: Oct. 3, 2023
(86) PCT No.: PCT/US2023/075849

§ 371 Date: Apr. 3, 2025

Related U.S. Application Data
(60) Provisional application No. 63/413,065, filed on Oct. 4, 2022.

(51) Int. Cl. A61K 31/53; A61P 25/22

(52) U.S. Cl. CPC A61K 31/53; A61P 25/22

(57) ABSTRACT

Described herein are uses of 2′,6-difluoro-5′-(3-(2-hydroxypropan-2-yl)imidazo[1,2-b][1,2,4]triazin-7-yl)-[1,1′-biphenyl]-2-carbonitrile or a salt thereof. Also described herein are compositions, such as dosage forms, of 2′,6-difluoro-5′-(3-(2-hydroxypropan-2-yl)imidazo[1,2-b][1,2,4]triazin-7-yl)-[1,1′-biphenyl]-2-carbonitrile or a salt thereof, and uses thereof.

### RELATED APPLICATIONS

This application claims the benefit of U.S. Provisional patent application 63/413,065 filed Oct. 4, 2022, the entire contents of which are incorporated by reference herein.

### BACKGROUND

Gamma-aminobutyric acid (GABA) receptors respond to the neurotransmitter GABA, which is the major inhibitory compound of the vertebrate central nervous system. GABA_{A} receptors occur in all organisms that have a nervous system. Modulation of GABA_{A} receptors may therefore be useful in therapeutically addressing diseases or disorders of the central nervous system. Undesired sedation may be a side effect of GABA_{A}-receptor therapeutic engagement. Furthermore, work in a phase II trial using a nonbenzodiazepine anxiolytic (i.e. 7-(1,1-dimethylethyl)-6-(2-ethyl-2H-1,2,4-triazol-3-ylmethoxy)-3-(2-fluorophenyl)-1,2,4-triazolo[4,3-b]pyridazine) was terminated due to toxicity in long term dosing studies. Accordingly, there is a need for development of GABA_{A}-receptor therapeutics that mitigate negative side effects, including concurrent sedation effects and toxicity, of therapeutic intervention.

### SUMMARY

Described herein are uses of 2′,6-difluoro-5′-(3-(2-hydroxypropan-2-yl)imidazo[1,2-b][1,2,4]triazin-7-yl)-[1,1′-biphenyl]-2-carbonitrile or a salt thereof. Also described herein are compositions, such as dosage forms, of 2′,6-difluoro-5′-(3-(2-hydroxypropan-2-yl)imidazo[1,2-b][1,2,4]triazin-7-yl)-[1,1′-biphenyl]-2-carbonitrile (Compound 1), or a pharmaceutically acceptable salt thereof, and uses thereof.

Compound 1, or its salts, may alternatively be referred to herein as an active compound or active ingredient. Compound 1 is shown below as a free base.

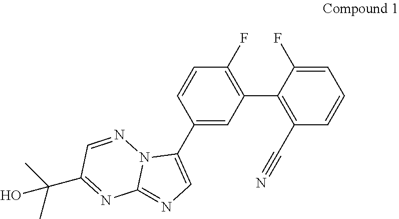 [SMILES: CC(C)(O)c1cnn2c(-c3ccc(F)c(-c4c(F)cccc4C#N)c3)cnc2n1]

### BRIEF DESCRIPTION OF THE DRAWINGS

FIG. 1 shows onset of REM, NREM, and latency of REM from Example 1.

FIG. 2 shows changes in delta spectral bands over time during NREM (0-12 h post dose) from Example 1, Frontal Cortex; Compound 1 and Lorazepam.

FIG. 3 shows changes in theta spectral bands over time during NREM (0-12 h post dose) from Example 1, Frontal Cortex; Compound 1 and Lorazepam.

FIG. 4 shows changes in alpha spectral bands over time during NREM (0-12 h post dose) from Example 1, Frontal Cortex; Compound 1 and Lorazepam.

FIG. 5 shows changes in beta spectral bands over time during NREM (0-12 h post dose) from Example 1, Frontal Cortex; Compound 1 and Lorazepam.

FIG. 6 shows changes in low-gamma spectral bands over time during NREM (0-12 h post dose) from Example 1, Frontal Cortex; Compound 1 and Lorazepam.

FIG. 7 shows changes in high-gamma spectral bands over time during NREM (0-12 h post dose) from Example 1, Frontal Cortex; Compound 1 and Lorazepam.

FIG. 8 shows changes in delta spectral bands over time during NREM (0-12 h post dose) from Example 1, Parietal Cortex; Compound 1 and Lorazepam.

FIG. 9 shows changes in beta spectral bands over time during NREM (0-12 h post dose) from Example 1, Parietal Cortex; Compound 1 and Lorazepam.

FIG. 10 shows changes in theta spectral bands over time during NREM (0-12 h post dose) from Example 1, Parietal Cortex; Compound 1 and Lorazepam.

FIG. 11 shows changes in low-gamma spectral bands over time during NREM (0-12 h post dose) from Example 1, Parietal Cortex; Compound 1 and Lorazepam.

FIG. 12 shows changes in alpha spectral bands over time during NREM (0-12 h post dose) from Example 1, Parietal Cortex; Compound 1 and Lorazepam.

FIG. 13 shows changes in high-gamma spectral bands over time during NREM (0-12 h post dose) from Example 1, Parietal Cortex; Compound 1 and Lorazepam.

FIG. 14 shows percent change in spectral bands during NREM from Example 1.

FIG. 17 shows Change from Baseline (CFB) EEG Alpha-power Fz-Cz Eyes Open ((uV)²).

FIG. 18 shows Change from Baseline EEG Beta-power Fz-Cz Eyes Closed ((uV)²).

FIG. 19 shows Change from Baseline EEG Theta-power Fz-Cz Eyes Closed ((uV)²).

FIG. 20 shows Change from Baseline EEG Theta-power Fz-Cz Eyes Open ((uV)²).

FIG. 21 shows Compound 1 significantly decreased SPV (p=<0.05*), consistent with an anxiolytic-like profile. *p-value refers to the entire dosing period of 12 days.

### DETAILED DESCRIPTION

### Definitions

Certain terms, whether used alone or as part of a phrase or another term, are defined below.

The articles “a” and “an” refer to one or to more than one of the grammatical object of the article.

Numerical values relating to measurements are subject to measurement errors that place limits on their accuracy. For this reason, the term “about” modifies all numerical values provided herein, unless otherwise indicated. The term “about” generally indicates a possible variation of no more than 10%, 5%, or 1% of a numerical value. In some embodiments, the last decimal place of a numerical value provided herein indicates its degree of accuracy. In some embodiments, where no other error margins are given, the maximum margin is ascertained by applying the rounding-off convention to the last decimal place or last significant digit when a decimal is not present in the given numerical value.

The term “amelioration” means a lessening of severity of at least one indicator of a condition or disease, such as a delay or slowing in the progression of one or more indicators of a condition or disease. The severity of indicators may be determined by subjective or objective measures which are known to those skilled in the art.

The terms “composition” and “pharmaceutical composition” refer to a mixture of at least one compound described herein with a carrier or a pharmaceutically acceptable carrier, respectively. The pharmaceutical composition facilitates administration of the compound to a patient or subject. Multiple techniques of administering a composition exist including, but not limited to, intravenous, oral, nasal, rectal, intravaginal, aerosol, parenteral, buccal, sublingual, ophthalmic, pulmonary, transdermal and topical administration.

The terms “effective amount” and “therapeutically effective amount” refer to an amount of therapeutic compound, such as a compound described herein, administered to a subject, either as a single dose or as part of a series of doses, which is effective to produce a desired therapeutic effect.

The term “pharmaceutically acceptable carrier” means a pharmaceutically acceptable material, composition or carrier, such as a liquid filler, solid filler, stabilizer, dispersing agent, suspending agent, diluent, excipient, thickening agent, solvent, or encapsulating material, involved in carrying or transporting at least one compound described herein within or to the patient such that the compound may perform its intended function. A given carrier must be “acceptable” in the sense of being compatible with the other ingredients of a particular formulation, including the compounds described herein, and not injurious to the patient. Other ingredients that may be included in the pharmaceutical compositions or dosage forms described herein are known in the art and described, for example, in “Remington's Pharmaceutical Sciences” (Genaro (Ed.), Mack Publishing Co., 1985), the entire content of which is incorporated herein by reference.

The term “pharmaceutically acceptable salt” refers to derivatives of the disclosed compounds wherein the parent compound is modified by converting an existing acid or base moiety to its salt form. Lists of salts are found in “Handbook of Pharmaceutical Salts: Properties, Selection, and Use” (P. Henrich Stahl & Camille G. Wermuth (Eds.), VHCA & Wiley-VCH, 2002), the entire content of which is incorporated herein by reference.

The term “solid form” includes, but is not limited to, polymorphs, crystalline forms, amorphous forms, solvates, and hydrates of a compound.

The terms “treatment” or “treating” refer to the application of one or more specific procedures used for the amelioration of a disease. A “prophylactic” treatment, refers to reducing the rate of progression of the disease or condition being treated, delaying the onset of that disease or condition, or reducing the severity of its onset.

Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise dearly contradicted by context. The use of any and all examples, or exemplary language (“for example,” “such as,” etc.) provided herein is intended merely to better illuminate the described subject matter and does not pose a limitation on the scope of the subject matter otherwise claimed. No language in the specification should be construed as indicating any non-claimed element essential to practicing the described subject matter.

Each group member of a grouping of alternative elements or embodiments of this disclosure may be referred to and claimed individually or in any combination with other members of the group or other elements found herein. Furthermore, a recited member of a group may be included in, or excluded from, another recited group for reasons of convenience or patentability.

Reference made to a patent or printed publication document throughout this specification incorporate herein by reference the document's entire content.

Embodiments of this disclosure are illustrative. Accordingly, the present disclosure is not limited to that precisely as shown and described.

### Dosage Forms

2′,6-difluoro-5′-(3-(2-hydroxypropan-2-yl)imidazo[1,2-b][1,2,4]triazin-7-yl)-[1,1′-biphenyl]-2-carbonitrile (Compound 1), or a pharmaceutically acceptable salt thereof, is a GABA_{A} receptor modulator that can act at the benzodiazepine site of the GABA_{A} receptor as a selective allosteric modulator of the α2, α3, and α5 subtypes. Compound 1, or its salts, as described herein are synthesized using any suitable procedures starting from compounds that are available from commercial sources, or may be prepared using procedures described in U.S. Pat. Nos. 6,936,608 or 6,617,326, or U.S. Patent Application Publication No. 2021/0040103. General methods for the preparation of a compound as described herein are modified by the use of appropriate reagents and conditions, for the introduction of the various moieties found in the formula as provided herein.

Provided herein are compositions, such as pharmaceutical compositions, such as dosage forms of Compound 1, or a pharmaceutically acceptable salt thereof, and uses thereof. In some embodiments, Compound 1 is provided as a phosphate salt of Compound 1. Dosages (mg/kg) used in rat studies can be extrapolated to human equivalent dosages by multiplying the rat dosage by a factor of 0.162 (J Basic Clin Pharm. March 2016-May 2016; 7(2): 27-31). Thus, in some embodiments, the dosage forms provided herein include about 1.5 to about 100 μg/mL of Compound 1 or its salt. In some embodiments, the dosage forms provided herein include about 15 to about 20 μg/mL of Compound 1 or its salt. In some embodiments, the dosage forms provided herein include about 3 μg/mL of Compound 1 or its salt. In some embodiments, the dosage forms provided herein include about 10 to about 100 μg/mL of Compound 1 or its salt. In some embodiments, the dosage forms provided herein include about 20 μg/mL of Compound 1 or its salt.

In some embodiments, the dosage forms provided herein may comprise a first pharmaceutically acceptable carrier. In some embodiments, the first pharmaceutically acceptable carrier includes methyl 2-hydroxyethyl cellulose. In some embodiments, the dosage forms include about 0.1 to about 3% w/w methyl 2-hydroxyethyl cellulose. In some embodiments, the dosage forms include about 0.5% or about 2% w/w methyl 2-hydroxyethyl cellulose. In some embodiments, the dosage forms provided herein further include, independently, a second, third, or more, pharmaceutically acceptable carrier. Beneficially, the pharmaceutically acceptable carrier, for example, methyl 2-hydroxyethyl cellulose, may enable Compound 1, or its salt, to remain in suspension in the dosage form longer than a composition that does not include methyl 2-hydroxyethyl cellulose. That is, by including methyl 2-hydroxyethyl cellulose in the dosage form may delay settling of suspended Compound 1, or its salt. Thus, the dosage forms provided herein may be stable to settling for up to 6 or 8 days when stored at room temperature (about 20 to about 25 °C). Undesirable settling of an active ingredient poses a number of problems, including risk of a lower amount of active ingredient drawn into or poured into a container when the dosage form is a liquid dosage form.

In some embodiments, the dosage forms provided herein include a dose of Compound 1, or its salt, of about 0.002 to about 1 mg/kg. In some embodiments, the dosage forms provided herein include a dose of Compound 1, or its salt, of about 0.002 to about 0.2 mg/kg. In some embodiments, the dosage forms provided herein include a dose of Compound 1, or its salt, of about 0.005 mg/kg, about 0.01 mg/kg, about 0.015 mg/kg, about 0.016 mg/kg, about 0.05 mg/kg, about 0.15 mg/kg, or about 0.16 mg/kg, or any range between these numerical values. In some embodiments, the dosage forms provided herein include a dose of Compound 1, or its salt, of about 0.01 to about 1 mg/kg. In some embodiments, the dosage forms provided herein include a dose of Compound 1, or its salt, of about 0.03 mg/kg, about 0.1 mg/kg, about 0.3 mg/kg, or about 1 mg/kg, or any range between these numerical values.

In some embodiments, the dosage forms include about 0.5, 1.0, 1.5, 2.0, 2.5, 3.0, 3.5, 4.0, 4.5, or 5.0 mg Compound 1 free base equivalent or Compound 1 phosphate. In some embodiments, the dosage forms herein include about 1, 2, 3, 4, 5, 6, 7, 8, 9, 10, 11, 12, 13, 14, 15, 20, 25, or 30 μmol Compound 1 or a salt thereof, e.g., a phosphate salt thereof.

In some embodiments, the dosage form is a liquid or solid dosage form. In some embodiments, the dosage form is an oral dosage form.

### Methods

Thus, in some embodiments, provided herein are methods, including administering a dosage form provided herein to a subject. In some embodiments, the subject suffers from a disease or condition that Compound 1, or its salts, is useful in treating.

The dosage forms described herein, which include Compound 1, and pharmaceutically acceptable salts thereof (e.g., Compound 1 phosphate), may be used as described in U.S. Pat. Nos. 6,936,608 or 6,617,326, or U.S. Patent Application Publication No. 2021/0040103. Thus, in some embodiments, the dosage forms provided herein are useful in treating certain disorders, including disorders of the central nervous system, in a subject in need thereof. In some embodiments, the dosage forms provided herein are useful in treating a GABA_{A} receptor related disease or a disease or disorder of the central nervous system in a subject in need thereof. In some embodiments, the dosage forms provided herein are useful in treating anxiety, a cognitive disorder, or convulsions in a subject in need thereof. In some embodiments, the dosage forms provided herein are useful in treating an adverse neurological condition in a subject in need thereof. In some embodiments, the dosage forms provided herein are useful in treating alcohol dependence, anxiety, autism (for example, autism resulting from SCN2a mutation, fragile X syndrome, or autism related to ion-channel dysfunction), cognitive impairment, depression, drug dependence, epilepsy (for example, Focal epilepsy, Generalized epilepsy, Dravet Syndrome, Childhood absence epilepsy (CEA), Juvenile absence epilepsy, Juvenile myoclonic epilepsy (JME), West Syndrome, Lennox-Gastaut syndrome (LGS), Sunflower Syndrome, Staticus epilepticus, Nerve agent induced seizures, Tremors from alcohol withdrawal, Traumatic Brain Injury, Tuberous Sclerosis Complex, Doose Syndrome, Rasmussen's Syndrome, Early myoclonic encephalopathy, Malignant migrating partial seizures of infancy, Epilepsy with continuous spike and waves during slow wave sleep, Landau-Kleffner syndrome, Benign epilepsy with centrotemporal spikes, Benign familial neonatal infantile seizures, Cortical dysplasia focal epilepsy syndrome, Generalized epilepsy with febrile seizure plus (GEFS+), Myoclonic atonic epilepsy, Malignant migrating partial seizures of infancy, Ohtahara syndrome (a.k.a. early infantile epileptic encephalopathy), or Partial epilepsy and febrile seizures plus), generalized anxiety disorder, itch (for example, chronic itch, neurogenic itch, uremic pruritus, neurodermatitis, notalgia paresthetica, atopic dermatitis, prurigo nodularis, psoriasis, psychogenic itch or aquagenic itch), muscle spasms, pain (for example, fibromyalgia, inflammatory pain, neuropathic pain, peripheral diabetic neuropathy, chemotherapy induced pain, HIV associated neuropathy, post-herpetic neuralgia, musculoskeletal pain, rheumatoid arthritis, osteoarthritis, post-operative pain, burn pain, sunburn pain, or phantom limb pain), panic disorder, pruritus, or schizophrenia in a subject in need thereof.

Additional disorders that the dosage forms described herein are useful in treating include anxiety disorders, such as panic disorder with or without agoraphobia, agoraphobia without history of panic disorder, animal and other phobias including social phobias, social anxiety disorder, obsessive-compulsive disorder, stress disorders including post-traumatic and acute stress disorder, and generalized or substance-induced anxiety disorder; neuroses; convulsions; migraine; depressive or bipolar disorders, for example single-episode or recurrent major depressive disorder, dysthymic disorder, bipolar I and bipolar II manic disorders, and cyclothymic disorder; psychotic disorders including schizophrenia; neurodegeneration arising from cerebral ischemia; attention deficit hyperactivity disorder; speech disorders, including stuttering; and disorders of circadian rhythm, for example, in subjects suffering from the effects of jet lag or shift work. In some embodiments, the anxiety disorder is post-traumatic stress disorder (PTSD).

Other disorders for which the dosage forms provided herein may be of benefit include pain and nociception; emesis, including acute, delayed and anticipatory emesis, in particular emesis induced by chemotherapy or radiation, as well as motion sickness, and post-operative nausea and vomiting; eating disorders including anorexia nervosa and bulimia nervosa; premenstrual syndrome; muscle spasm or spasticity, for example, in paraplegic patients; hearing disorders, including tinnitus and age-related hearing impairment; urinary incontinence; and the effects of substance abuse and dependency, including alcohol withdrawal. The dosage forms provided herein may also be effective as pre-medication prior to anesthesia or minor procedures such as endoscopy, including gastric endoscopy.

In some embodiments, provided herein are methods comprising administering to a subject in need thereof a therapeutically effective amount of a dosage form herein that includes Compound 1 phosphate.

In some embodiments, provided herein are methods wherein the subject does not experience a decrease in time to non-REM onset of sleep as compared to a subject receiving a corresponding dose (e.g., equimolar) of lorazepam.

In some embodiments, provided herein are methods comprising administering to a subject in need thereof a therapeutically effective amount of 2′,6-difluoro-5′-(3-(2-hydroxypropan-2-yl)imidazo[1,2-b][1,2,4]triazin-7-yl)-[1,1′-biphenyl]-2-carbonitrile or a pharmaceutically acceptable salt thereof, wherein the subject does not experience a decrease in time to non-REM onset of sleep as compared to a subject receiving a corresponding dose of lorazepam.

In some embodiments of the methods herein, delta, theta, or delta and theta electroencephalogram (EEG) oscillation power from the subject are decreased by about 20% or more during non-REM sleep as compared to a subject receiving a corresponding dose of lorazepam.

In some embodiments of the methods herein, the delta, theta, or delta and theta EEG oscillation power is measured at the frontal, parietal, or frontal and parietal cortex of the subject's brain.

In some embodiments of the methods herein, the subject suffers from a GABA_{A} receptor related disease or a disease or disorder of the central nervous system. In some embodiments, the disease comprises anxiety. In some embodiments, the disease comprises general anxiety disorder. In some embodiments, the disease is a CNS disorder. In some embodiments, the disease comprises epilepsy.

In some embodiments, provided herein are methods of treating a GABA_{A} receptor related disease or a disease or disorder of the central nervous system in a subject in need thereof, comprising administering to the subject a therapeutically effective amount of a dosage form herein, optionally wherein the subject does not experience a decrease in time to non-REM onset of sleep as compared to a subject receiving a corresponding dose of lorazepam.

In some embodiments, provided herein are methods of treating a GABA_{A} receptor related disease or a disease or disorder of the central nervous system in a subject in need thereof, comprising administering to the subject a therapeutically effective amount of 2′,6-difluoro-5′-(3-(2-hydroxypropan-2-yl)imidazo[1,2-b][1,2,4]triazin-7-yl)-[1,1′-biphenyl]-2-carbonitrile phosphate, wherein the subject does not experience a decrease in time to non-REM onset of sleep as compared to a subject receiving a corresponding dose of lorazepam.

In some embodiments, provided herein are methods comprising administration of a dosage form herein to a subject, the method further comprising generation of a pharmacokinetic parameter for the compound in a subject, wherein: the administration of the dosage form is once per day; the dosage form comprises about 0.5, 1.0, 1.5, or 2.0 mg of 2′,6-difluoro-5′-(3-(2-hydroxypropan-2-yl)imidazo[1,2-b][1,2,4]triazin-7-yl)-[1,1′-biphenyl]-2-carbonitrile phosphate; and the pharmacokinetic parameter is a Tₘₐₓ of about 3 hours or a T_{1/2} of about 60-70 hours, or both.

In some embodiments, provided herein are methods comprising administration of a dosage form herein to a subject, the method further comprising generation of a pharmacokinetic parameter for the compound in a subject, wherein: the administration of the dosage form is once per day; the dosage form comprises about 1, 2, 3, 4, or 5 pmol of 2′,6-difluoro-5′-(3-(2-hydroxypropan-2-yl)imidazo[1,2-b][1,2,4]triazin-7-yl)-[1,1′-biphenyl]-2-carbonitrile phosphate; and the pharmacokinetic parameter is a Tₘₐₓ of about 3 hours or a T_{1/2} of about 60-70 hours, or both.

In some embodiments, provided herein are methods of treating an anxiety disorder in a subject in need thereof, comprising administration of a dosage form herein to the subject.

In some embodiments of these methods, the administration is oral once daily administration.

In some embodiments of these methods the dosage form comprises 1, 2, or 3 tablets or capsules comprising the compound.

In some embodiments of these methods the dosage form comprises about 1.0, 1.3, 2.0, 2.6, 3.0, 3.1, 3.8, 4.0, 4.1, 5.0, or 5.1 μmol of 2′,6-difluoro-5′-(3-(2-hydroxypropan-2-yl)imidazo[1,2-b][1,2,4]triazin-7-yl)-[1,1′-biphenyl]-2-carbonitrile or 2′,6-difluoro-5′-(3-(2-hydroxypropan-2-yl)imidazo[1,2-b][1,2,4]triazin-7-yl)-[1,1′-biphenyl]-2-carbonitrile mono-phosphate.

In some embodiments of these methods the dosage form comprises about 2 mg of 2′,6-difluoro-5′-(3-(2-hydroxypropan-2-yl)imidazo[1,2-b][1,2,4]triazin-7-yl)-[1,1′-biphenyl]-2-carbonitrile or 2′,6-difluoro-5′-(3-(2-hydroxypropan-2-yl)imidazo[1,2-b][1,2,4]triazin-7-yl)-[1,1′-biphenyl]-2-carbonitrile mono-phosphate.

In some embodiments of these methods 2′,6-difluoro-5′-(3-(2-hydroxypropan-2-yl)imidazo[1,2-b][1,2,4]triazin-7-yl)-[1,1′-biphenyl]-2-carbonitrile or a pharmaceutically acceptable salt thereof is present in plasma of the subject at a steady-state when undergoing once daily administration of 2′,6-difluoro-5′-(3-(2-hydroxypropan-2-yl)imidazo[1,2-b][1,2,4]triazin-7-yl)-[1,1′-biphenyl]-2-carbonitrile phosphate, and the once daily administration includes at least 6 consecutive days of administration.

In some embodiments of these methods, the steady-state in plasma of the subject includes a Cₘₐₓ of the compound of about 10-50 ng/mL.

In some embodiments, provided herein are methods of treating anxiety (e.g., general anxiety disorder) in a subject in need thereof, comprising administration of a dosage form herein (e.g., one including 1, 2, 3, 4, 5, 6, 7, 8, 9, 10, 11, 12, or 13 μmol Compound 1 or Compound 1 phosphate, or an equivalent amount in milligrams) to the subject, wherein: the administration of the dosage form is once per day; the dosage form comprises about 1, 2, 3, 4, 5, 6, 7, 8, 9, 10, 11, 12, or 13 μmol of 2′,6-difluoro-5′-(3-(2-hydroxypropan-2-yl)imidazo[1,2-b][1,2,4]triazin-7-yl)-[1,1′-biphenyl]-2-carbonitrile phosphate; the once daily administration includes at least 12 consecutive days of administration of the dosage form; and the subject experiences less mydriasis from administration of the dosage form as compared to once daily administration of the dosage form for 1, 2, 3, 4, 5, 6, 7, 8, 9, 10, or 11 consecutive days.

Actual dosage levels of Compound 1, or its salts, that is, the active compound, in the dosage forms described herein may be varied so as to obtain an amount of the active ingredient that is effective to achieve the desired therapeutic response for a particular patient, composition, and mode of administration, without being toxic to the patient.

In particular, the selected dosage level will depend upon a variety of factors including the activity of the particular active ingredient employed, the time of administration, the rate of excretion of the active ingredient, the duration of the treatment, other drugs, compounds or materials used in combination with the active ingredient, the age, sex, weight, condition, general health, and prior medical history of the patient being treated.

Routes of administration of include, without limitation, oral, nasal, rectal, intravaginal, aerosol, parenteral, buccal, sublingual, ophthalmic, pulmonary, and topical administration. In some embodiments, the oral or nasal route of administration is an oral inhalational or nasal inhalational route of administration. The dosage forms for use as described herein may be formulated for administration by any suitable route to achieve the particular method being applied.

Accordingly, administration of a dosage form described herein includes a variety of enteral or parenteral approaches selected from, without limitation: oral administration in any acceptable form, for example, tablet, liquid (for example, liquid suspension of particles), capsule, powder, or the like; topical or transdermal administration in any acceptable form, including, for example, drops, spray, creams, gels ointments, or patches; buccal, nasal, sublingual, ophthalmic, pulmonary, and/or inhalation administration in any acceptable form; rectal administration in any acceptable form; vaginal administration in any acceptable form; peri- and intra-tissue administration in any acceptable form, including, for example, intraperitoneal injection, intramuscular injection, subcutaneous injection, intravenous injection, or intraarticular injection; intravesicular administration in any acceptable form, including, for example, catheter instillation; and by placement device, including, for example, an implant, a stent, a patch, a pellet, a catheter, an osmotic pump, a suppository, a bioerodible delivery system, a non-bioerodible delivery system or another implanted extended or slow release system.

Local administration results in significantly more delivery of an active ingredient to a specific location as compared to the entire body of the mammal, whereas, systemic administration results in delivery of an active ingredient to essentially the entire body of the individual. Routes of administration suitable for or treating a central nervous system related disease or disorder as disclosed herein also include both central and peripheral administration. Central administration results in delivery of an active ingredient to essentially the central nervous system of the individual and includes, for example, nasal administration, intrathecal administration, epidural administration as well as a cranial injection or implant. In some embodiments, central administration is used to administer the dosage forms described herein.

Central administration by the nasal route, which targets drug absorption through the vascular plexus of the nasal cavity, is distinct from administration by nasal inhalation, which delivers drug through the pulmonary system. Whereas the latter typically uses liquid or dry powder aerosols with mean particle sizes less than about 10 microns, central administration may be accomplished using mean particle sizes of about 10 microns or larger. Mists and aerosols can be generated using nebulizers, dry powder inhalers, pressurized aerosols, and atomization pumps. It is also feasible to use nose drops (for example, a suspension of particles in a liquid) for central administration by the nasal route.

Peripheral administration results in delivery of an active ingredient to essentially any area of an individual outside of the central nervous system and encompasses any route of administration other than direct administration to the spine or brain.

### Kits

In some embodiments, provided herein are packaged dosage forms, comprising a container holding a therapeutically effective amount of Compound 1 or its salt, and instructions for using the dosage form in accordance with one or more of the methods provided herein.

The present dosage forms and associated materials can be finished as a commercial product by the usual steps performed in the present field, for example by appropriate sterilization and packaging steps. For example, the material can be treated by UV/vis irradiation (200-500 nm), for example using photo-initiators with different absorption wavelengths (for example, Irgacure 184, 2959), preferably water-soluble initiators (for example, Irgacure 2959). Such irradiation is usually performed for an irradiation time of 1-60 min, but longer irradiation times may be applied, depending on the specific method. The material according to the present disclosure can be finally sterile-wrapped so as to retain sterility until use and packaged (for example, by the addition of specific product information leaflets) into suitable containers (boxes, etc.).

According to further embodiments, the described dosage forms can also be provided in kit form combined with other components necessary for administration of the material to the patient. For example, disclosed kits, such as for use in the treatments described herein, can further comprise, for example, administration materials.

The kits may be designed in various forms based on the specific deficiencies they are designed to treat.

The dosage forms provided herein may be prepared and placed in a container for storage at ambient or elevated temperature. For example, it was found that solutions of Compound 1 or its salt at about 20 μg/mL in water, or about 0.12 to about 7.2 mg/mL in about 0.5% methyl 2-hydroxyethyl cellulose in water, were stable (that is, no appreciable degradation was observed) for up to about 6 to about 8 days when stored at room temperature (about 20 to about 25 °C). This is beneficial because transportation of commercially viable dosage forms may benefit from stability at temperatures greater than those requiring refrigeration or sub-freezing environments during transportation and storage at the site of use.

When the dosage forms provided herein are stored in a polyolefin plastic container as compared to, for example, a polyvinyl chloride plastic container, discoloration of the dosage form may be reduced. Without being bound by theory, the container may reduce exposure of the container's contents to electromagnetic radiation, whether visible light (for example, having a wavelength of about 380-780 nm) or ultraviolet (UV) light (for example, having a wavelength of about 190-320 nm (UV B light) or about 320-380 nm (UV A light)). Some containers also include the capacity to reduce adherence or adsorption of the active ingredient to the surface of the container, which could effectively dilute the concentration of active ingredient in the contained solution. Some containers also include the capacity to reduce exposure of the container's contents to infrared light, or a second component with such a capacity. Some containers further include the capacity to reduce the exposure of the container's contents to heat or humidity. The containers that may be used include those made from a polyolefin such as polyethylene, polypropylene, polyethylene terephthalate, polycarbonate, polymethylpentene, polybutene, or a combination thereof, especially polyethylene, polypropylene, or a combination thereof. In some embodiments, the container is a glass container. The container may further be disposed within a second container, for example, a paper container, cardboard container, paperboard container, metallic film container, or foil container, or a combination thereof, to further reduce exposure of the container's contents to UV, visible, or infrared light. Articles of manufacture benefiting from reduced discoloration, decomposition, or both during storage, include dosage forms that include Compound 1 or its salts. The dosage forms provided herein may need storage lasting up to, or longer than, three months; in some cases up to, or longer than one year. The containers may be in any form suitable to contain the contents—for example, a bag, a bottle, or a box.

The following examples further illustrate embodiments of the present disclosure. However, they are in no way a limitation of the teachings or disclosure as described herein.

### EXAMPLES

### Example 1: Evaluation of Sleep-Stage Specific Pharmaco-EEG (Electroencephalography) Signatures of Compound 1 in Wirelessly Implanted Rats

Young adult male Sprague-Dawley (SD) rats (~275-325 grams on arrival) were used in the study. Animals implanted were used in study 1 and study 2. Upon receipt, rats were housed with 3 rats per cage. During the course of the study, 12/12 light/dark cycles were maintained. The room temperature was maintained between 20° and 23 °C with a relative humidity maintained around 50%. Chow and water were provided ad libitum for the duration of the study. Following surgery rats were single housed. After a period of recovery (7-10 days), animals were transferred to an electroencephalogram (EEG) recording room, and placed over a dry sensor interface (DSI) receiver for recordings.

### Study 1.

Compound 1 was administered orally (per os (PO)) at 0.03 mg/kg, 0.1 mg/kg, 0.3 mg/kg, or 1 mg/kg in 0.5% TYLOSE MH 300® solution (supplied as 2% methyl 2-hydroxyethyl cellulose viscosity 150-450 mPa*s (centipoise, cP) in water). Formulations were prepared daily by weighing the test item, grinding the test item to a fine powder using a mortar and pestle, adding a few drops of vehicle, mixing with the test item to obtain a homogeneous mixture, progressively adding the vehicle and transferring the mixture into a gauged flask, rinsing the mortar and pestle and adding the rinsing liquid into the gauged flask, and homogenizing the suspension by manual stirring before completing to final volume with vehicle. The formulations were agitated by magnetic stirring at room temperature before any sampling. Standard solutions at 20 μg/mL were found to be stable for 6 days when they are stored at room temperature. Analytical samples diluted from dose formulations at concentrations ranging from 0.12 to 7.2 mg/mL in 0.5% TYLOSE MH 300® solution are found to be stable for 8 days when they are stored at room temperature. Compound was protected from light (for example, in an opaque bottle such as plastic or glass) when not in use.

Vehicle and 4 doses of Compound 1 were assessed in a cross-over design to ensure n=12 rats are obtained in each group. After four days of washout, recordings were performed on a Monday and Friday dosing/recording schedule. EEG Recordings start at about 6 PM (at time of lights OFF). Dosing occurs at about 10 PM (4 hours after lights OFF). EEG recordings are continuous for 48 hours.

Animals were implanted with DSI Telemetry devices (F50-EET) in a 3 channel (lead pairs) configuration. Each of the EEG channels on the DSI transmitter functions as a differential input whereby the differential input measures the difference between the positive and negative leads. Frontal/Parietal (right): (+) 2 mm Anterior, 2 mm Lateral; (−) 4 mm Posterior, 2 mm Lateral; Frontal/Frontal (bihemispheric): (+) 2 mm Anterior, 2 mm Lateral (right); (−) 2 mm Anterior, 2 mm Lateral (left); and neck EMG.

EEG was recorded wirelessly from rats in their home cages using DSI DataQuest ART. Light cycle was maintained at 12 hours lights on and 12 hours lights off for the duration of the experiment. Animals were habituated to dosing (vehicle dosing) for 2 days before any data collection takes place. Recordings were started 2-hour prior to compound administration and recorded continuously for 48 hours after administration.

Raw EEG recordings were manually scored using Neuroscore software (Data Sciences International) to identify sleep stages: Active Wake, Quiet Wake, NREM and REM.

Using NeuroScore (DSI), artifacts were removed offline from the data and sleep stages assigned manually for every 10 s epoch using EEG, EMG and Locomotor activity (LMA) by conventional methods as previously described (Ivarsson et al., 2005; Parmentier-Batteur et al., 2012; Leiser et al., 2014, 2015) using the fronto-parietal EEG, LMA and EMG: active wake (less regular, low-amplitude EEG with high EMG and LMA activity); quiet wake (less regular, low-amplitude EEG, with low EMG and no LMA activity); NREM (consisting of high-amplitude irregular waves with predominant delta (1-4 Hz), low EMG and no LMA); REM sleep (stable, low-amplitude waves dominated by theta (4-8 Hz) with near absent EMG and no LMA).

The sleep stage data were exported from a Neuroscore report template of sleep state time per each 15 min (2 hours pre-dose to 4 hours post-dose). The onset of the first sleep and first REM were also reported directly from a Neuroscore report template. Latency was calculated as the time period from the onset of the first REM bout following the onset of NREM (i.e. TREM−TNREM=REM Latency). Hypnograms were prepared using the percent time spent in each sleep stage per hour time bins. Time in each sleep state was calculated as a percent of total time in each sleep state (mean f standard error of the mean, SEM). The data for individual animals were arranged by treatment group, sleep state, bin and exported to GraphPad PRISM for statistics and graph illustration.

Spectral analysis was performed using Matlab. In summary, the time domain signal collected for multiple channels was collected into DSI/Neuroscore and then the EDF files were transferred to Matlab. Excel files, marking specific timestamps of baseline and post dose, were also transferred to Matlab for time-locking. In Matlab, the power spectral density (PSD) was computed using the Welch method. Next, both raw and relative spectral power was computed for each of the six frequency bands (Delta, Theta, Alpha, Beta, Low-Gamma and High Gamma) and for each 1 Hz sub band. 2 hours of data recorded prior to compound dosing were pooled and defined as “baseline.” Percent change from baseline was calculated based on each channel, subject, dose level, and spectral band and time segment. The average raw, relative and percent change for each frequency band were calculated for each group. Spectral analysis included quantifying the raw, relative, and percent change spectral power for the traditionally defined EEG bands (Delta (0.5-3.9 Hz), Theta (4-7.9 Hz), Alpha (8-11.9 Hz), Beta (12-29.9 Hz), Low Gamma (30-50 Hz), and High Gamma (51-100 Hz)) per recording per rat. Additionally, 1-100 Hz EEG spectra were represented in line plots.

### Study 2.

Lorazepam, 1 mg/kg, intraperitoneal (IP), was dissolved in saline. Following Study 1, all rats (n=12) receive a single dose of Lorazepam. These data were incorporated into Study 1 and analyzed the same way.

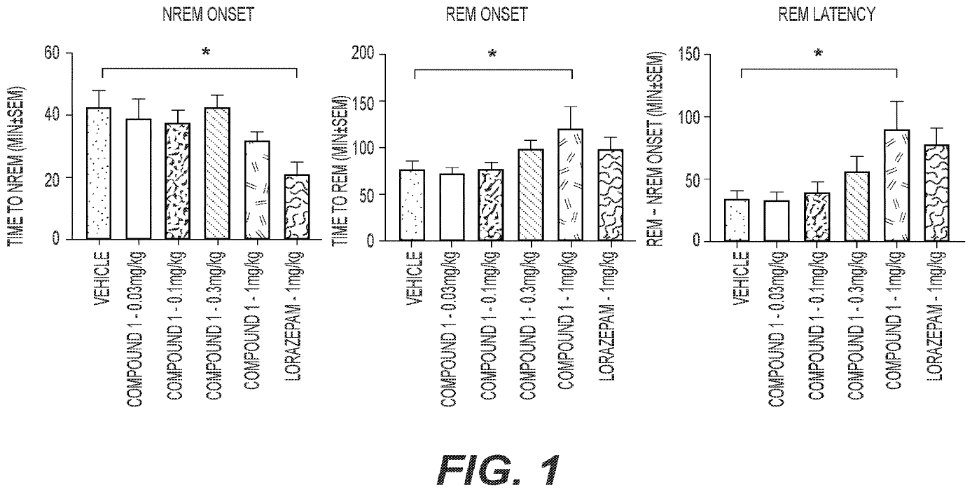

### Sleep Results (See FIG. 1)

1 mg/kg Compound 1 delayed the onset of REM and increased REM latency. Compound 1 and Lorazepam decreased Active Wake but only Compound 1 decreased Quiet Wake as well. Compound 1 and Lorazepam increased NREM.

FIG. 1 shows onset of REM, NREM, and latency of REM. Onset is the time (minutes) when the first bout of REM begins. Latency is defined as the time to onset of the first REM bout following the onset of NREM (i.e. TREM−TNREM=REM Latency). *p<0.05; ANOVA with Dunnett's multiple comparison's test.

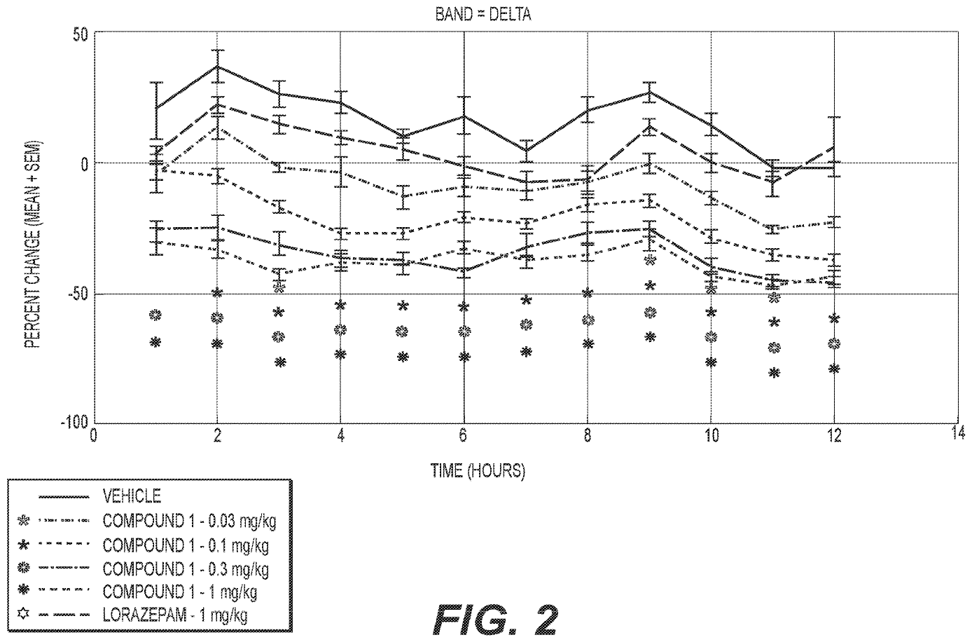

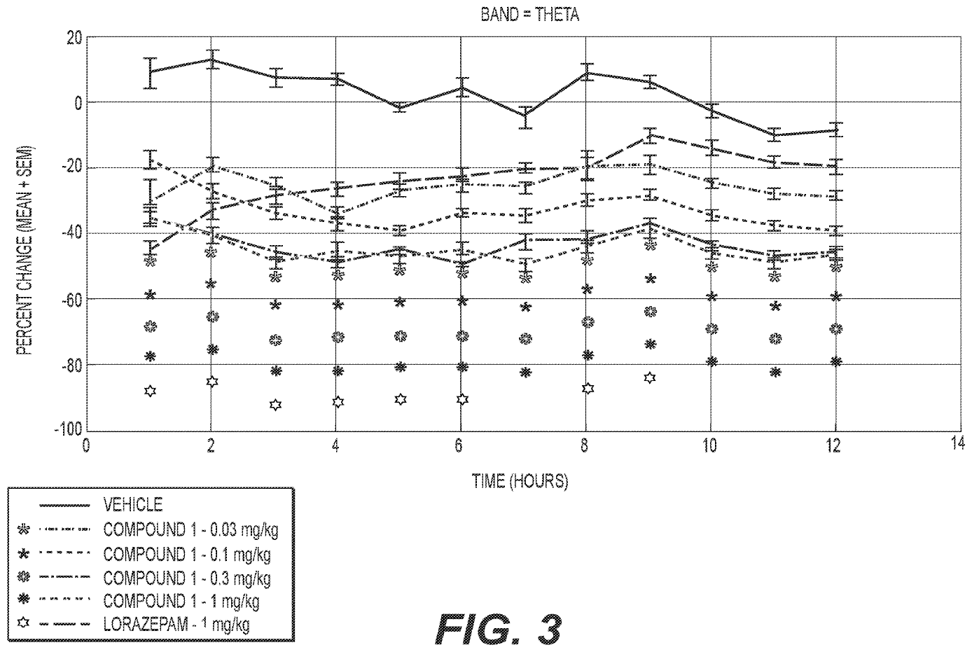

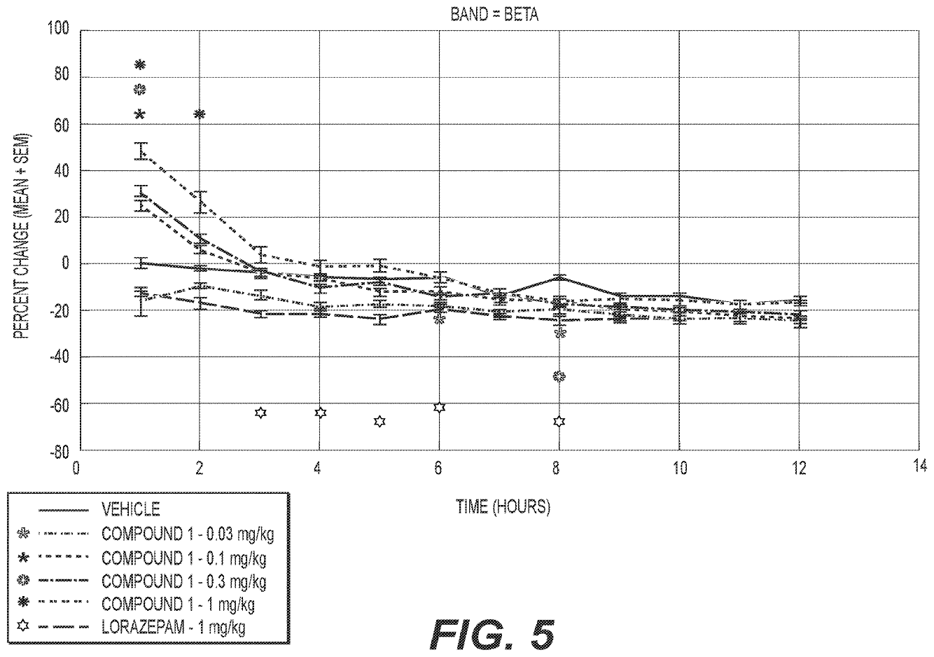

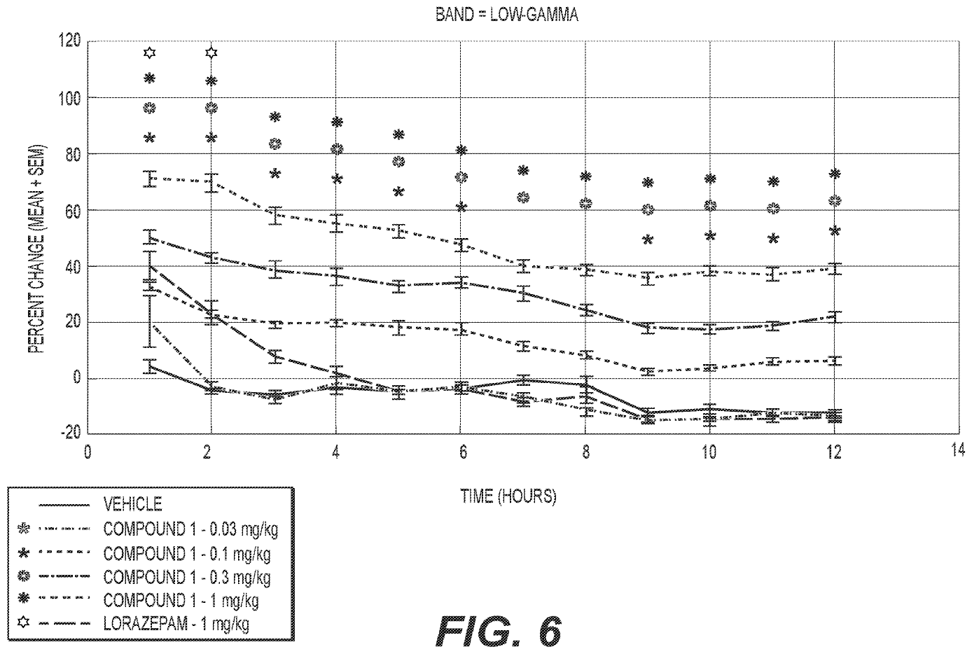

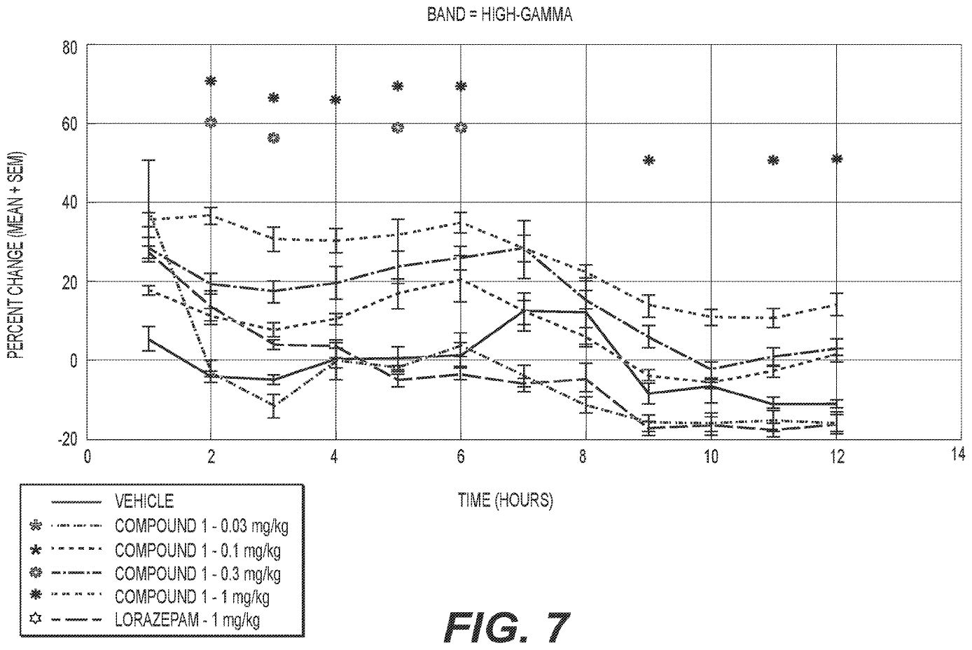

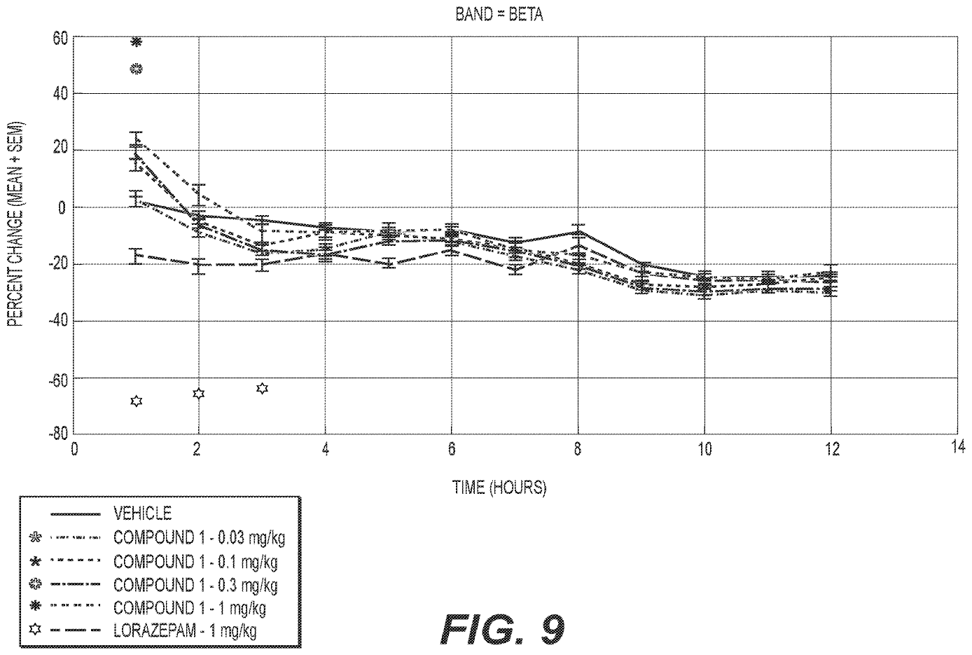

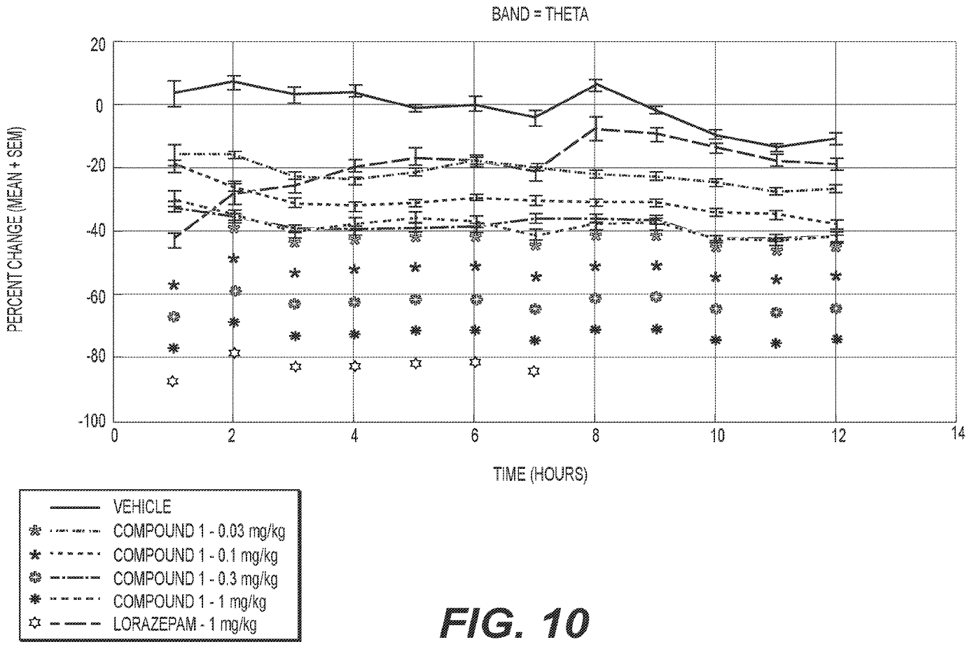

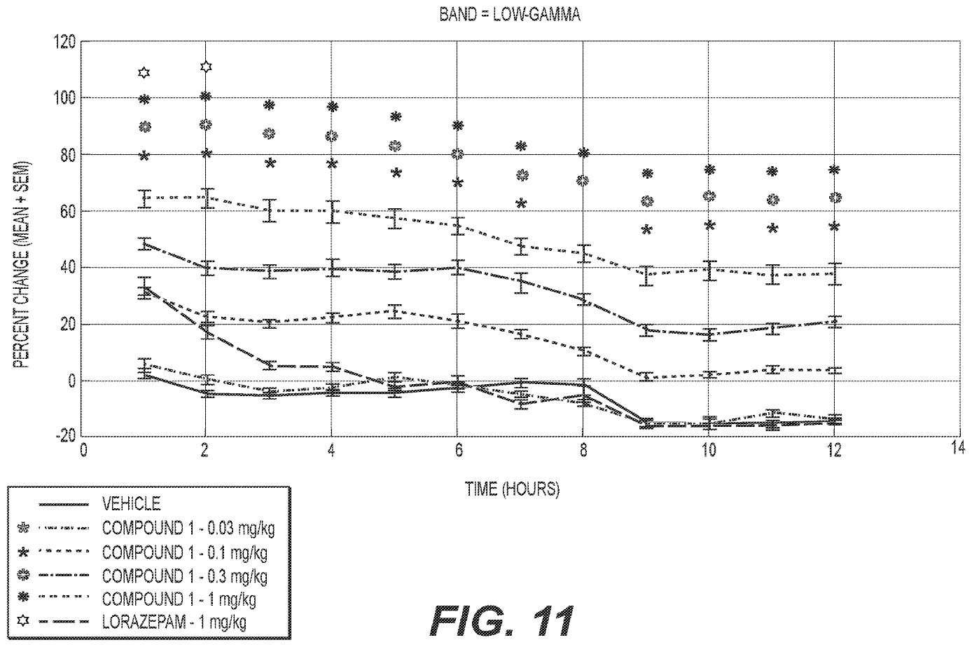

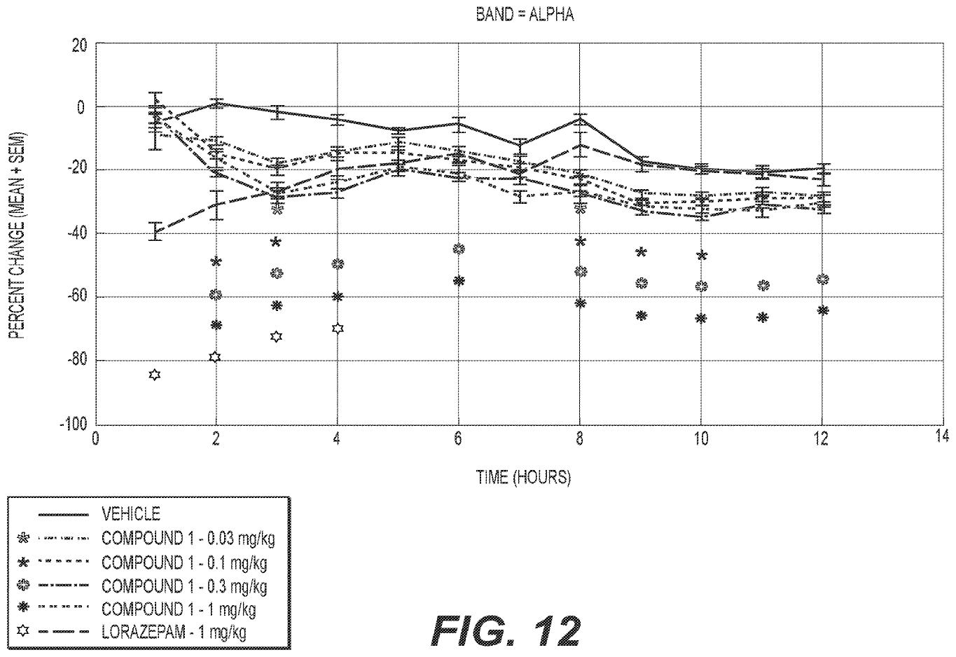

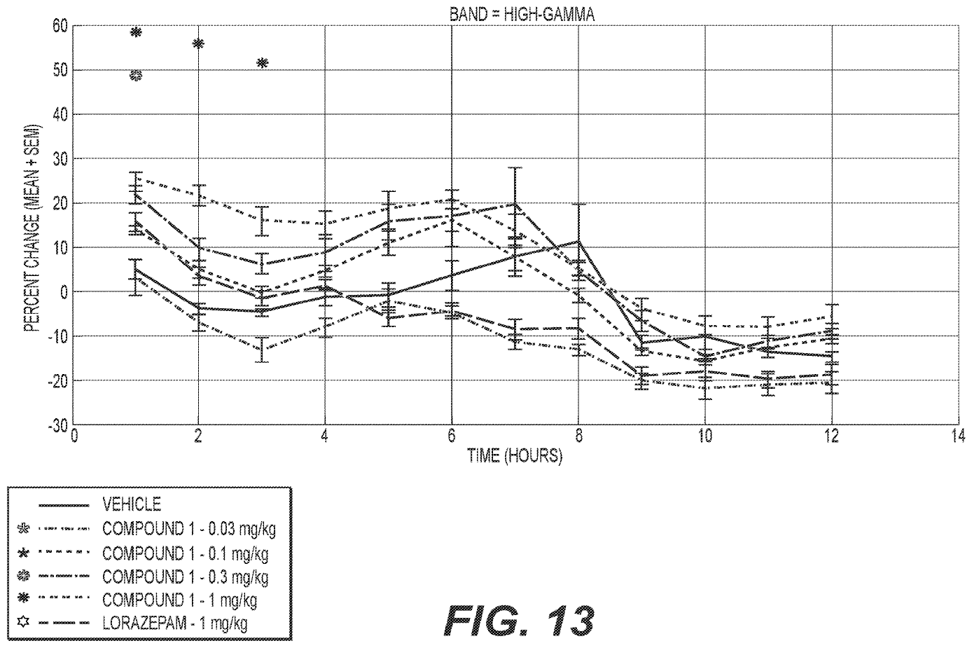

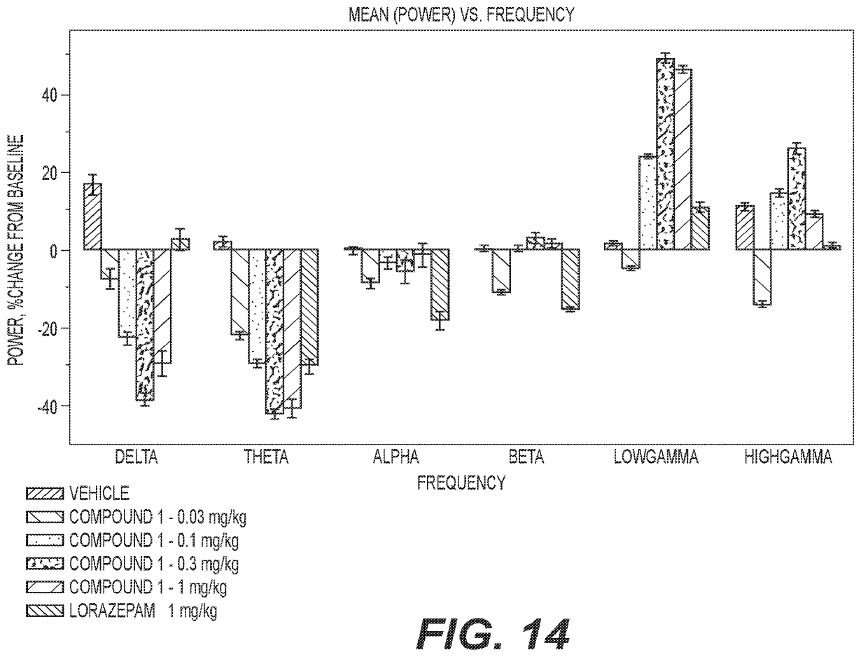

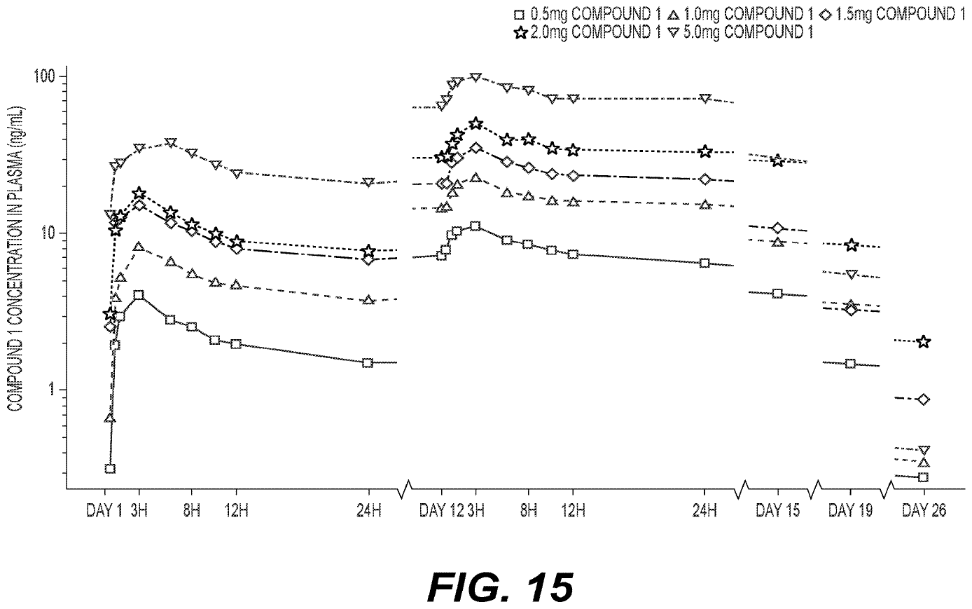

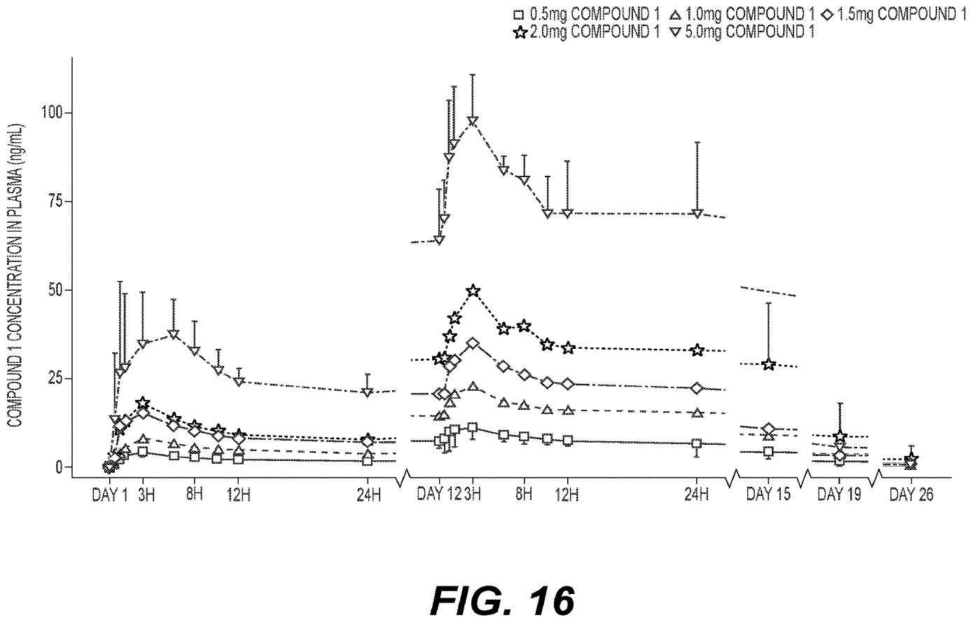

### Spectral Results (See FIGS. 2-14)

Frontal-Frontal cortex: Compound 1 and Lorazepam increased beta, low and high gamma during Active and Quiet Wake. Compound 1 and Lorazepam both increased beta during REM. Compound 1 and Lorazepam both decreased theta and alpha during NREM. Compound 1 also decreased delta and increased low and high gamma during NREM, effects lasting to 48 hours at the high dose, whereas Lorazepam decreased theta, alpha, and beta, but increased low gamma with effects lasting to 9 hours post dose.

Frontal-Parietal cortex: Compound 1 decreased alpha but increased beta, low and high gamma during Active Wake. Compound 1 decreased delta but increased beta, low and high gamma during Quiet Wake with effects lasting to 48 hours. Lorazepam decreased theta, increased beta and low and high gamma but the effects did not last past four hours post dose. During REM, Compound 1 decreased theta and alpha but increased beta and low gamma whereas Lorazepam increased theta, alpha, beta, and low gamma, but these effects did not last as long. Compound 1 decreased delta, theta, and alpha during NREM with effects in delta and theta lasting to 48 hours post dose. An increase in low and high gamma was observed with Compound 1 lasting to 48 hours but Lorazepam only lasting to two hours post dose.

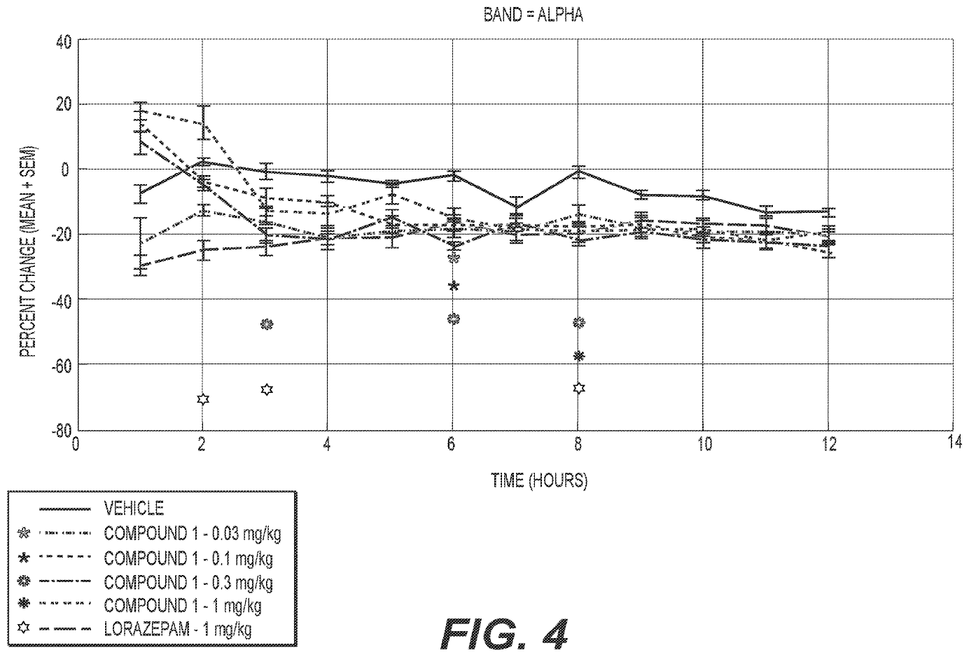

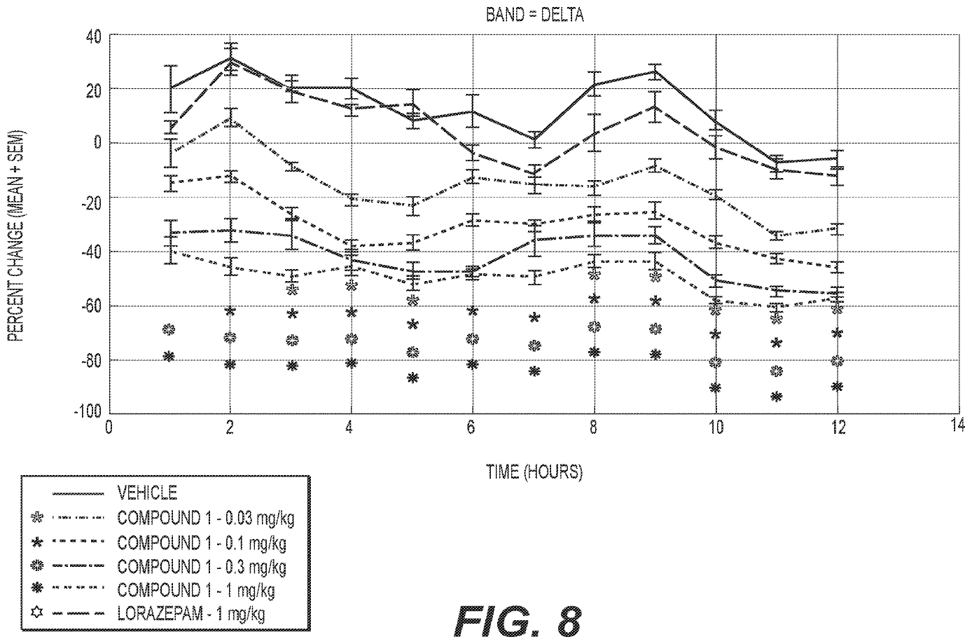

FIGS. 2-7 shows changes in spectral bands over time during NREM (0-12 h post dose; Frontal Cortex; Compound 1 and Lorazepam), FIGS. 8-13 shows spectral bands over time during NREM (0-12 h post dose; Parietal Cortex; Compound 1 and Lorazepam), and FIG. 4 shows percent change in spectral bands during NREM. FIGS. 2-7 and FIGS. 8-13 identify percent change from baseline EEG power bands over time for each frequency at each sleep state in the Parietal cortices after treatment with Compound 1 or Lorazepam. Pre-dose values were averaged from 2 hours baseline recording and post-dose values were calculated in 60-minute bins up to 24 hours. Shown here is the percent change from baseline value for each of the frequency bands displayed over the 24-hour post dose period. Statistics: *p<0.05 relative to vehicle, ANOVA and Dunnett's multiple comparisons test.

### SUMMARY

Compound 1 (1 mg/kg) did not change NREM onset but decreased latency to REM onset. REM Latency, which is the time to REM from first Non-REM, also decreased. Lorazepam decreased the latency to NREM onset.

During Quiet Wake, Compound 1 increased beta and low gamma. Lorazepam increased low & high gamma, decreased delta. During Active Wake, Compound 1 increased low gamma and decreased alpha (Parietal ctx). During Non-REM, Compound 1 suppressed power in low frequencies (Delta, Theta, and Alpha) and increased higher frequencies (gamma). During REM, Compound 1 increased beta.

Lorazepam induces a quicker time to Non-REM onset sleep than Compound 1 (FIG. 1). Compound 1 does not decrease time to Non-REM onset (FIG. 1). This is important because Non-REM is the first stage of sleep (light sleep).

During Non-REM sleep, Compound 1 induces significant decreases in delta and theta power across all doses (ANOVA w Dunnett's multiple comparisons test). This can be seen plotted in FIG. 4 where 40% decreases in delta/theta are reported during Non-REM sleep. FIGS. 2-7 (frontal cortex) and FIGS. 8-13 (parietal cortex) also depict the decreases in delta and theta power induced by Compound 1 dose-dependently and in relationship to lorazepam.

### Example 2: Preclinical Evaluation of Compound 1, a GABA_{A} α2,3,5 PAM that Blocks α1, in the Elevated Plus Maze and Pharmaco-EEG

Background: Nonselective gamma-aminobutyric acid type A (GABA_{A}) positive allosteric modulators (PAMs) such as benzodiazepines, are proven anxiolytics, but their clinical use is limited due to significant side effects, largely mediated by activation of α1 subunit-containing GABA_{A} receptors. There have been several efforts to generate subtype selective (α2, α3, or α5 subunits) GABA_{A} modulators driving the beneficial effects of GABA modulation without the undesired effects that result from the activation of α1 subunit-containing receptors.

Compound 1 is a GABA_{A} PAM that activates neurotransmission via GABA_{A} receptors containing α2, α3, and α5 subunits, while blocking α1. The potential for anxiolysis of Compound 1 was evaluated in rodent anxiety models such as the elevated plus maze (EPM) after acute and chronic dosing, and the sleep-wake electroencephalography (EEG) profile was characterized in rats induced by increasing doses of Compound 1.

Methods: The in vitro pharmacology of Compound 1 was assessed with the SyncroPatch platform in PAM mode on the human GABA_{A} receptors α1/β3/γ2 α2/β2/γ2, α2/β3/γ2, α3/β3/γ2, and α5/β3/γ2. Adult male SD rats (N=130; N=10 per condition) were used in the in vivo pharmacology studies. Compound 1 was evaluated in the EPM test which assesses anxiety. The following measures were automatically recorded: distance travelled, time spent in each arm, and entries into each arm. Compound 1 was orally dosed at 0.1, 0.3 and 1 mg/kg to target receptor occupancies of approximately 30%, 50% and 80% at 1 hr post-dosing, respectively. Acutely dosed rats were also tested 24 hrs post-dose. A different cohort was dosed chronically, once daily for fourteen days and tested 1 hr following the last dose to assess the anxiolytic properties of Compound 1 after continuous target engagement. Chlordiazepoxide was used as a positive control. Data were analyzed by ANOVA followed by post hoc analyses where appropriate.

Adult male SD rats (N=12 in a cross-over design) were used in the pharmaco-EEG studies. Sleep-stage specific pharmaco-EEG signature of Compound 1 was assessed after orally dosing Compound 1 (0.03 mg/kg, 0.1 mg/kg, 0.3 mg/kg and 1 mg/kg) to target a broad range of receptor occupancies. Lorazepam (1 mg/kg) was used as a positive control. Rats were implanted with telemetry devices. Recordings started 2 hrs prior to compound administration and recorded continuously for 48 hrs after administration. Spectral analysis included quantifying the changes in spectral power for the traditional EEG bands (Delta 0.5-3.9 Hz, Theta 4-7.9 Hz, Alpha 8-11.9 Hz, Beta 12-29.9 Hz, Low Gamma 30-49.9 Hz, and High Gamma 50-100 Hz).

Sleep analysis was also conducted for 2 hrs prior to Compound 1 administration and for 12 hours after Compound 1, as well as 23-25 hrs and 46-48 hrs after Compound 1 was administered. Several variables were analyzed: sleep architecture as percent time in active awake, quiet wake, NREM, REM and latency to sleep. ANOVA with Dunnett's multiple comparisons was applied independently to each 15-minute bin to assess the drug effects on the power of different EEG spectral bands as well as across sleep stages.

All experimental protocols in animal studies were approved by the Institutional Animal Care and Use Committee and were conducted in accordance with the National Institutes of Health Guide for the Care and Use of Laboratory Animals.

Results: The functional in vitro activity of Compound 1 on GABA_{A} receptors showed that Compound 1 is a subtype-specific GABA_{A} PAM. Compound 1 did not drive Cl-flux on cloned human GABA_{A} subtype α1/β3/γ2. Compound 1 displayed EC₅₀ of 0.80 nM, 0.82 nM, 2.47 nM and 0.20 nM at GABA_{A} subtypes α2/β2/γ2, α2/β3/γ2, α3/β3/γ2, and α5/β3/γ2, respectively; and Eₘₐₓ of 50%, 89%, 113% and 96% at GABA_{A} subtypes α2/β2/γ2, α2/β3/γ2, α3/β3/γ2, and α5/β3/γ2, respectively.

In vivo characterization of Compound 1 in the EPM assay showed that Compound 1 increased distance traveled in the open arms demonstrating an anxiolytic-like profile. The effect was equivalent to what was demonstrated by the reference anxiolytic compound, chlordiazepoxide. Chronic dosing of Compound 1 showed a similar magnitude of anxiolytic effect. These data demonstrate strong anxiolytic activity in rodents that is maintained with chronic dosing.

The EEG profile induced in rats by dosing Compound 1 was also studied. Compound 1 induced EEG changes indicative of an anxiolytic profile and that mimic the spectral EEG signature that has been associated to other subtype-selective GABA modulators. This spectral EEG signature can be used as an indication of central pathway engagement in rodents and as a translational bridge into clinical studies.

Conclusions: Compound 1 is anxiolytic-like in rodents after acute and sub-chronic (14 days) dosing without any loss of effect despite engaging the target for the entire duration of the study. Sleep-wake pattern and EEG profile analysis suggest a non-sedative anxiolytic profile of Compound 1. These data support the dinical study of Compound 1 in anxiety-related indications.

### Example 3: A Multiple Ascending Dose Study to Evaluate Safety, Tolerability, Pharmacokinetics, and Pharmacodynamics of Compound 1 at Plasma Steady-State in Healthy Volunteers

TABLE 1
Dosage Forms.

| | Compound 1 | | Placebo | |
|---|---|---|---|---|
| | Strengths* | | | |
| | 0.5 mg | 2.0 mg | 0.5 mg matched | 2.0 mg matched |
| | Route | | | |
| | Oral | | | |
| Formulation | Size 4 rich | Size 3 rich | Size 4 rich | Size 3 rich |
| | yellow opaque | yellow opaque | yellow opaque | yellow opaque |
| | hard gelatin | hard gelatin | hard gelatin | hard gelatin |
| | capsule | capsule | capsule | capsule |

*Compound 1 provided as Compound 1•H₂PO₄. Using a conversion factor of 1.25, 0.5 mg Compound 1 free base corresponds to about 0.63 mg Compound 1•H₂PO₄, and 2.0 mg Compound 1 free base corresponds to about 2.5 mg Compound 1•H₂PO₄.

TABLE 2
QD Treatment Regimens.

| Cohort (dose) | Regimen (Compound 1 or placebo) |
|---|---|
| 1 (0.5 mg) | 12 days of 1 × 0.5 mg capsule QD |
| 2 (1.0 mg) | 12 days of 2 × 0.5 mg capsules QD |
| 3 (1.5 mg) | 12 days of 3 × 0.5 mg capsules QD |
| 4 (2.0 mg) | 12 days of 1 × 2.0 mg capsule QD |
| 5 (5.0 mg) | 11 days of 2 × 0.5 mg and 2 × 2.0 mg capsules QD |
| | and 1 day of 10 × 0.5 mg capsules QD |

GABA is the major fast inhibitory neurotransmitter in humans fulfilling much of its role through activation of the GABA_{A} receptor. GABA_{A} receptors are ligand-gated chloride channels comprised of 5 protein subunits (a mixture of α1-6, β1-3, γ1-3, δ, ε, θ, π). GABA_{A} receptors that contain σ1, σ2, σ3, or α5 subunits in combination with βx subunits and a γ2 subunit in a 2:2:1 (α:β:γ) ratio form an allosteric binding site separate from where GABA binds. This site, termed the benzodiazepine binding site, enables positive allosteric modulation of the ion channel. Nearly 90% of GABA_{A} receptors in the CNS contain a benzodiazepine binding site and those that do are classified into subtypes by their α subunit. The expression patterns of α-subtypes vary across the CNS, thereby differentiating their pharmacology.

Without being bound by theory, gamma-aminobutyric acid (GABA_{A}) modulation is one pharmacologic approach for the treatment of epilepsy, anxiety, and other central nervous system (CNS) disorders. However, long-term therapeutic benefit by non-selective pharmacological agents is limited due to a rapid loss of efficacy and significant side effects including sedation, dizziness, and ataxia. Side effects of non-selective GABA_{A} agonists may result primarily from GABA_{A} α1-subtype activity. Compound 1 is an α2/α3/α5-subtype-selective positive allosteric modulator that does not potentiate the α1 GABA_{A} receptor subtype and, therefore, may be effective, well tolerated, and usable long-term for the treatment of epilepsy, anxiety, or other CNS disorders.

A clinical study was developed to evaluate safety, tolerability, pharmacokinetics, and pharmacodynamics of Compound 1 at plasma steady-state in 40 healthy human volunteers. Compound 1 (provided as Compound 1 phosphate) was administered to participants as multiple ascending doses of 0.5 mg (N=6), 1.0 mg (N=6), 1.5 mg (N=6), 2.0 mg (N=6), or 5.0 mg (N=6) once daily (QD) for 12 days.

### Safety Results

Safety and tolerability of Compound 1 were assessed through listings and summaries by Compound 1 dose versus placebo (observed and change from baseline where applicable) for the following measures: treatment-emergent adverse events (TEAEs), clinical laboratory evaluation, vital signs, electrocardiogram, physical examination, pregnancy test, C-SSRS, and MOAA/S (Modified Observer's Assessment Alertness/Sedation). Continuous safety data were summarized with descriptive statistics (n, arithmetic mean, standard deviation (SD), median, minimum, and maximum) by dose level. Categorical safety data were summarized with incidence counts and percentages by dose level.

All TEAEs were transient in nature and required no treatment or intervention.

No severe TEAEs or serious adverse events (SAEs) occurred. The majority of participants (34/40; 85.0%) experienced TEAEs of mild severity, while 6 TEAEs of moderate severity occurred in 5/40 participants (12.5%).

The most commonly reported TEAEs were somnolence and fatigue. The incidence of somnolence in Compound 1 Cohorts was approximately twice as high as the placebo-treated participants pooled across all Cohorts.

There was no overall clear relationship between increase in dose and frequency and/or nature of TEAEs.

The mean changes in clinical laboratory values and vital signs were similar across all treatment Cohorts, including placebo.

No clinically relevant increase in mean QT corrected for heart rate by Fridericia's cube root formula (QTcF) was observed.

No consistent differences were observed between treatments for use of concomitant medications or physical examination findings.

No positive responses were provided for queries regarding suicidal ideation or behaviors as per C-SSRS in any of the participants after any treatment.

No clear difference from placebo in sedation across ascending Cohorts according to objective MOAA/S (Modified Observer's Assessment Alertness/Sedation) scoring was observed. A decrease in the MOAA/S score for alertness/sedation indicates impaired participant alertness and increased sedation as objectively determined by a clinician. Treatment with Compound 1 resulted in little to no impairment of alertness and no frank sedation. For Compound 1 5.0 mg, a MOAA/S score of 2 (“Responds only after mild prodding or shaking”) was assessed for 1 participant. For the other participants in the Compound 1 5.0 mg Cohort and all other Cohorts, MOAA/S scores were above 3 indicating no difference from placebo in sedation across ascending Cohorts.

### Pharmacokinetic Results

Blood samples were collected, and plasma concentrations were measured using a validated high performance liquid chromatography/liquid chromatography-mass spectrometry/mass spectrometry method. The PK parameters assessed included Cₘₐₓ, Tₘₐₓ, AUC from administration to the end of dosing (AUC₀₋ₜ), AUC₀₋₂₄, AUC_{0-inf}, t_{1/2}, terminal rate constant (λz), apparent total clearance of the drug from plasma (CL/F), and apparent volume of distribution during terminal phase after non-intravenous administration (Vz/F).

The plasma concentration data for Compound 1 were summarized by dose level and the planned sampling time points using descriptive statistics (n, SD, median, minimum, maximum, geometric mean, and coefficient of variance (CV %) for the geometric mean) for the PK Analysis Set. Dose proportionality for Compound 1 based on area-under-the-curve from time 0 to infinity, (AUC_{0-inf}) (Day 1), area-under-the-curve from time 0 to 24 h (AUC₀₋₂₄) (Day 12), and maximum concentration (Cₘₐₓ) (Day 1 and Day 12) values from Cohorts 1, 2, 3, 4, and 5 on a log scale were examined using a model with fixed effects for the log-dose and time points (Day 1 or Day 12) and log-dose by Day interaction and random participant effect.

Compound 1 was rapidly absorbed, with a median time to maximum concentration (Tₘₐₓ) varying from 3 h up to 2.0 mg to 4.5 h at 5.0 mg on Day 1.

Mean Cₘₐₓ and AUC₀₋₂₄ generally increased dose- and concentration-proportionally.

Steady-state was reached by 6 to 12 days of repeated dosing.

Compound 1 followed bi-exponential decline with an apparent terminal half-life (t_{1/2})±SD ranging between 39±9 h for 5.0 mg and 66±24 h for 0.5 mg.

Variability in key PK parameters was acceptable up to 5.0 mg administered repeatedly for 12 days.

Specifically, samples for PK analyses were taken on Day 1 and Day 12 pre-dose, 0.5, 1, 1.5, 3, 6, 8, 10, and 12 h post-dose, with additional samples taken on every dosing Day (24 hours after the previous day's dose) and 24, 72, 168, and 336 h after last dosing on Day 12. All individual Compound 1 concentrations were above the LLOQ of 0.0500 ng/mL at 0.5 h post-dose, except for 1 participant, for whom the concentration was above the LLOQ at 1 h post-dose.

The PK profile of Compound 1 followed a bi-exponential decline, as seen by the clear distinction of 2 different slopes in the decreasing part of the PK profile on a log-linear scale (FIG. 15). The absorption and distribution phase ranged from Tₘₐₓ (median of 3 h) until approximately 12 h, after which the elimination phase commenced. An overview of PK parameters of Compound 1 at all dose levels is presented in Table 3 and a visualization of the mean plasma concentrations is presented in FIG. 16.

In summary:
Median Tₘₐₓ occurred at 3.00 h on both Day 1 and Day 12 at all doses, except for Compound 1 5.0 mg, at which the maximum concentration on Day 1 occurred at median 4.50 h. The median Tₘₐₓ did not evidently change with ascending doses (Table 3);
Mean Cₘₐₓ±SD increased dose-proportionally over the investigated dose range:
   On Day 1, mean Cₘₐₓ±SD was 4.6±2 ng/mL, 8.5±3 ng/mL, 18.8±7 ng/mL, 19.6±4 ng/mL, and 49.7±13 ng/mL for Compound 10.5 mg, 1.0 mg, 1.5 mg, 2.0 mg, and 5.0 mg, respectively (Table 3);
   On Day 12, mean Cₘₐₓ±SD was 11.7±4 ng/mL, 23.3±5 ng/mL, 41.7±5 ng/mL, 49.8±17 ng/mL, and 99.1±14 ng/mL for 0.5 mg, 1.0 mg, 1.5 mg, 2.0 mg, and 5.0 mg, respectively (Table 3);
AUC increased dose-proportionally over the investigated dose range:
   Mean±SD AUC₀₋₂₄ was 53±7 ng*h/mL, 118±27 ng*h/mL, 217±48 ng*h/mL, 244±48 ng*h/mL, and 642±125 ng*h/mL for 0.5 mg, 1.0 mg, 1.5 mg, 2.0 mg, and 5.0 mg, respectively, on Day 1 (Table 3);
   Mean±SD AUCₜₐᵤ was 198±51 ng*h/mL, 410±115 ng*h/mL, 706±84 ng*h/mL, 873±331 ng*h/mL, and 1804±242 ng*h/mL for 0.5 mg, 1.0 mg, 1.5 mg, 2.0 mg, and 5.0 mg, respectively, on Day 12 (Table 3);
The highest individual AUC₀₋₂₄ and AUCₜₐᵤ reached was at 5.0 mg with 853.4 ng*h/mL and 2171.8 ng*h/mL, respectively (Table 3);
Dose-proportional PK were confirmed over the investigated dose range on both the Cₘₐₓ and AUCₜₐᵤ parameters;
Accumulation of Compound 1 to steady-state was observed, with a mean accumulation ratio±SD ranging from 3±0.3 to 4±0.7;
There was no trend towards change in t_{1/2} or λz with increasing dose;
Mean (±SD) t_{1/2} was 66±24 h, 61±29 h, 51±18 h, 61±26 h, and 39±9 h for Compound 1 0.5 mg, 1.0 mg, 1.5 mg, 2.0 mg, and 5.0 mg, respectively);
In the Compound 1 5.0 mg Cohort the PK sampling timepoint at 72 h post-dose was removed to decrease participant burden. Estimated t_{1/2} could have been affected by this;
Based on the average concentration at end of the dosing interval, steady-state plasma concentration was reached after 6 to 12 days of dosing with Compound 1;
PK variability was acceptable with coefficient of variations ranging between 12% and 36%, 12% and 38%, and 23% and 48% for Cₘₐₓ at Day 12, AUCₜₐᵤ, and t_{1/2}, respectively;
Last quantifiable concentration determined directly from individual concentration-time data ranged between 0.1 ng/mL and 9.5 ng/mL;
Median time of the last quantifiable concentration was approximately 24 h for all Cohorts on Day 1 and ranged from 73 to 384 h on Day 12, respectively
AUC_{0-inf}, CL/F, Vz/F, and dose normalized AUC_{0-inf} for Day 1 could not be calculated, as t_{1/2} for Day 1 could not be derived due to the elimination phase not being adequately captured prior to the next dose; and
Dose normalized Cₘₐₓ and AUC₀₋₂₄ were consistent with linear kinetics, with the arguable exception of Compound 1 1.5 mg, at which the median dose normalized Cₘₐₓ was higher on Day 1 and Day 12 and median dose normalized AUC₀₋₂₄ was higher on Day 1.

TABLE 3

| | | | | | | | | | |
|---|---|---|---|---|---|---|---|---|---|
| Day | Param. | Unit | N | Median | Mean | SD | CV % | Min | Max |

Compound 1 0.5 mg

| | | | | | | | | | |
|---|---|---|---|---|---|---|---|---|---|
| Day 1 | Cₘₐₓ | ng/mL | 6 | 4.58 | 4.615 | 1.6207 | 35.1 | 2.51 | 7.11 |
| | Tₘₐₓ | h | 6 | 3.00 | — | — | — | 1.82 | 6.00 |
| | AUC₀₋₂₄ | ng*h/mL | 6 | 54.8 | 52.56 | 6.738 | 12.8 | 43.7 | 61.3 |
| Day 12 | Cₘₐₓ | ng/mL | 5 | 11.60 | 11.660 | 4.2484 | 36.4 | 7.55 | 18.00 |
| | AUCₜₐᵤ | ng*h/mL | 5 | 201.3 | 197.54 | 50.534 | 25.6 | 137.1 | 264.5 |
| | Tₘₐₓ | h | 5 | 3.00 | — | — | — | 1.02 | 3.00 |
| | t_{1/2} | h | 5 | 64.35 | 65.610 | 23.6782 | 36.1 | 32.37 | 96.14 |

Compound 1 1.0 mg

| | | | | | | | | | |
|---|---|---|---|---|---|---|---|---|---|
| Day 1 | Cₘₐₓ | ng/mL | 6 | 8.99 | 8.497 | 2.6517 | 31.2 | 5.05 | 11.70 |
| | Tₘₐₓ* | h | 6 | 3.00 | — | — | — | 1.50 | 6.00 |
| | AUC₀₋₂₄ | ng*h/mL | 6 | 117.2 | 117.81 | 27.318 | 23.2 | 82.6 | 151.0 |
| Day 12 | Cₘₐₓ | ng/mL | 6 | 22.55 | 23.333 | 5.4213 | 23.2 | 17.20 | 29.60 |
| | AUCₜₐᵤ | ng*h/mL | 6 | 415.8 | 410.07 | 114.752 | 28.0 | 273.6 | 544.9 |
| | Tₘₐₓ | h | 6 | 3.00 | — | — | — | 1.50 | 3.05 |
| | t_{1/2} | h | 6 | 58.17 | 60.876 | 29.2396 | 48.0 | 27.94 | 113.50 |

Compound 1 1.5 mg

| | | | | | | | | | |
|---|---|---|---|---|---|---|---|---|---|
| Day 1 | Cₘₐₓ | ng/mL | 6 | 18.25 | 18.833 | 6.9058 | 36.7 | 11.00 | 29.00 |
| | Tₘₐₓ* | h | 6 | 3.00 | — | — | — | 1.00 | 6.00 |
| | AUC₀₋₂₄ | ng*h/mL | 6 | 226.7 | 216.99 | 48.171 | 22.2 | 158.2 | 272.3 |
| Day 12 | Cₘₐₓ | ng/mL | 5 | 42.60 | 41.700 | 4.8990 | 11.7 | 34.00 | 47.20 |
| | AUCₜₐᵤ | ng*h/mL | 5 | 737.6 | 705.65 | 84.204 | 11.9 | 557.6 | 767.1 |
| | Tₘₐₓ | h | 5 | 3.00 | — | — | — | 1.50 | 3.00 |
| | t_{1/2} | h | 5 | 49.06 | 50.526 | 17.7754 | 35.2 | 29.14 | 73.72 |

Compound 1 2.0 mg

| | | | | | | | | | |
|---|---|---|---|---|---|---|---|---|---|
| Day 1 | Cₘₐₓ | ng/mL | 6 | 19.95 | 19.633 | 3.9581 | 20.2 | 14.10 | 23.60 |
| | Tₘₐₓ* | h | 6 | 3.00 | — | — | — | 1.50 | 6.00 |
| | AUC₀₋₂₄ | ng*h/mL | 6 | 225.9 | 243.73 | 48.247 | 19.8 | 195.0 | 314.0 |
| Day 12 | Cₘₐₓ | ng/mL | 6 | 48.20 | 49.817 | 17.2081 | 34.5 | 28.20 | 74.50 |
| | AUCₜₐᵤ | ng*h/mL | 6 | 890.7 | 873.17 | 331.256 | 37.9 | 434.7 | 1404.7 |
| | Tₘₐₓ | h | 6 | 3.00 | — | — | — | 1.50 | 3.00 |
| | t_{1/2} | h | 6 | 52.30 | 61.167 | 26.4394 | 43.2 | 38.09 | 112.54 |

Compound 1 5.0 mg

| | | | | | | | | | |
|---|---|---|---|---|---|---|---|---|---|
| Day 1 | Cₘₐₓ | ng/mL | 6 | 49.55 | 49.700 | 13.0807 | 26.3 | 30.60 | 67.60 |
| | Tₘₐₓ* | h | 6 | 4.50 | — | — | — | 1.00 | 6.15 |
| | AUC₀₋₂₄ | ng*h/mL | 6 | 608.7 | 641.92 | 125.189 | 19.5 | 528.7 | 853.4 |
| Day 12 | Cₘₐₓ | ng/mL | 5 | 106.00 | 99.120 | 13.4591 | 13.6 | 79.50 | 112.00 |
| | AUCₜₐᵤ | ng*h/mL | 5 | 1800.2 | 1803.45 | 242.257 | 13.4 | 1537.3 | 2171.8 |
| | Tₘₐₓ | h | 5 | 3.00 | — | — | — | 1.00 | 3.05 |
| | t_{1/2} | h | 5 | 37.14 | 38.695 | 8.7849 | 22.7 | 30.32 | 53.55 |

Abbreviations: AUC₀₋₂₄ = area under the concentration-time curve from time 0 to 24 h; AUCₜₐᵤ = area under the concentration-time curve during the time interval between consecutive dosing events; Cₘₐₓ = maximum concentration; CV % = coefficient of variation; Min = minimum; Max = maximum; PK = pharmacokinetic; SD = standard deviation; Tₘₐₓ = time to maximum concentration; t_{1/2} = half-life; *= Tₘₐₓ is presented as median, min and max.

### Summary of Pharmacokinetic Findings

Compound 1 was rapidly absorbed, with a median Tₘₐₓ varying from 3 h up to 2.0 mg to 4.5 h at 5.0 mg on Day 1.

Mean Cₘₐₓ and AUC₀₋₂₄ generally increased dose- and concentration-proportionally.

Steady-state was reached by 6 to 12 days of repeated dosing.

Compound 1 followed bi-exponential decline with an apparent terminal t_{1/2}±SD ranging between 39±9 h for 5.0 mg and 66±24 h for 0.5 mg.

Variability in key PK parameters was acceptable up to 5.0 mg administered repeatedly for 12 days.

### Pharmacodynamic Results

Pharmacodynamic analyses included the NEUROCART® assessments, cognitive assessments Visual Verbal Learning Task (VVLT), and quantitative electroencephalography (qEEG). All PD analyses were based on the PD Analysis Set. For all PD endpoints, the baseline was calculated as average of the Day −1 measurements.

NEUROCART®, VVLT, and qEEG assessments were listed and summarized (absolute and change from baseline) using descriptive statistics at baseline and at each post-baseline time point. To establish whether significant treatment effects could be detected on the repeatedly measured NEUROCART® parameters, each parameter was analyzed with a mixed model analysis of covariance (ANCOVA) with treatment, time, and treatment by time as fixed factors, participant as a random factor, and the (average) baseline measurement as a covariate.

Unexpectedly, Compound 1 reduced saccadic peak velocity (SPV) to a comparable extent at 1.0 mg, 2.0 mg, and 5.0 mg on Day 1, an effect that persisted up to Day 12 and supports a sustained effect on arousal during repeated dosing.

Although Compound 1 decreased adaptive tracking at 21.0 mg and significantly increased body sway at 5.0 mg on Day 1, that was not the case for either on Day 12, indicating an acute effect on sustained attention that subsided over time and lack of effect on overall psychomotor function and postural balance at doses<5.0 mg during repeated dosing.

Compound 1 had no consistent effect on visual analogue scale (VAS) alertness. Compound 1 significantly decreased VAS alertness at 2.0 mg on Day 1 but not on Day 12, while VAS alertness at 5.0 mg remained unaffected on Day 1 and decreased significantly on Day 12.

Compound 1 had no consistent psychotomimetic or autonomic nervous system effects.

Compound 1 had no consistent effect on the WLT, though Compound 1 at 5.0 mg negatively affected memory encoding, retrieval, and consolidation on Day 1. This effect had dissipated by Day 12.

Compound 1 demonstrated the most consistent effects on qEEG at doses>1.5 mg, with decreased alpha- and theta-power on Day 12 that were suggestive of dose- or concentration-dependence and target engagement, but the increased beta-power on Day 12 did not suggest dose- or concentration-dependence.

NEUROCART® assessment summary. Compound 1 administered orally repeatedly up to 12 days:
Significantly decreased Saccadic Peak Velocity SPV at 1.0 mg, 2.0 mg, and 5.0 mg on both Days 1 and 12. Overall, no clear dose-dependent effect on SPV was seen. The decreases on Day 1 did not return to baseline, either 24 h post-dose or on Day 12, indicating a sustained effect on arousal during repeated dosing up to 12 days;
Significantly increased saccadic reaction time only at the highest dose level of 5.0 mg on Day 1, but not on Day 12;
Significantly decreased smooth pursuit eye movements at 0.5 mg, 1.5 mg, and 5.0 mg on Day 1 but not on Day 12;
Significantly decreased adaptive tracking at doses of ≥1.0 mg on Day 1 but not on Day 12, indicating an acute effect on sustained attention that subsided over time during repeated dosing up to 12 Days;
Significantly increased body sway at the highest dose level of 5.0 mg on Day 1 but not on Day 12, indicating an acute effect on psychomotor function and postural balance that subsided over time during repeated dosing up to 12 days. The absence of significant effects at dose levels<5.0 mg indicates the lack of effect on overall psychomotor function and postural balance;
Significantly decreased VAS alertness at 2.0 mg on Day 1 but not on Day 12, while VAS alertness at 5.0 mg remained unaffected on Day 1 but decreased significantly on Day 12; no significant decrease at other dose levels was observed;
Did not affect VAS mood or VAS calmness at any dose on either Day 1 or Day 12;
Demonstrated an inconsistent effect on Bowdle VAS: VAS external perception and VAS internal perception remained unaffected at 1.0 mg, 2.0 mg, and 5.0 mg but significantly increased at 1.5 mg on Day 1 and Day 12, while VAS feeling high significantly increased at 0.5 mg on Day 1 and Day 12, and at 1.5 mg on Day 12 with no apparent dose-related effect;
Significantly reduced left pupil/iris ratio at 1.0 mg, 1.5 mg, and 5.0 mg on Day 1 but not on Day 12, indicating mydriasis emerging following the first dose that subsided over time during repeated dosing up to 12 days; and
Had less consistent effects on SPV, body sway, VAS internal perception, VAS external perception, and VAS feeling high in the 1.5 mg Cohort compared to other dose levels.

Visual Verbal Learning Task summary. Compound 1 administered orally for 12 days:
Significantly increased reaction time for delayed word recognition at 1.0 mg on Day 12, but not on Day 1;
Significantly increased reaction time for delayed word recognition at 1.5 mg on Day 1, but not on Day 12;
Significantly decreased delayed word recognition but not immediate recall or delayed recall at 2.0 mg on Day 12, but not on Day 1; and
Significantly decreased immediate recall and decreased delayed recall/recognition at 5.0 mg on Day 1, but not on Day 12.

Compound 1 demonstrated inconsistent effects on reaction time for delayed word recognition up to 1.5 mg. Compound 1 2.0 mg negatively affected memory encoding but did not affect memory consolidation or retrieval on Day 12, whereas Compound 1 5.0 mg negatively affected memory encoding, retrieval, and consolidation on Day 1. The latter dissipated by Day 12, which supports tolerance to such effects following repeated dosing. Overall, Compound 1 had no consistent effect on WLT parameters.

Quantitative electroencephalography summary. Compound 1 administered orally for 12 days demonstrated the following effects on the qEEG:
Alpha-power:
   Significant decrease in Fz-Cz (eyes dosed) at 1.5 mg, 2.0 mg, and 5.0 mg on Day 12;
   Significant decrease in Fz-Cz (eyes open) at 1.5 mg on Day 1 and at 1.5 mg, 2.0 mg, and 5.0 mg on Day 12;
   Significant decrease in Pz-O1 (eyes closed) at 1.5 mg on Day 1 and at 1.5 mg, 2.0 mg, and 5.0 mg on Day 12;
   Significant decrease in Pz-O1 (eyes open) at 1.5 mg, 2.0 mg, and 5.0 mg on Day 12;
   Significant decrease in Pz-O2 (eyes dosed) at 1.5 mg, 2.0 mg, and 5.0 mg on Day 12; and
   Significant decrease in Pz-O2 (eyes open) at 1.5 mg, 2.0 mg, and 5.0 mg on Day 12;
Beta-power:
   Significant increase in Fz-Cz (eyes closed) at 2.0 mg and 5.0 mg on Day 1 but at none of the doses on Day 12;
   Significant increase in Fz-Cz (eyes open) at 2.0 mg and 5.0 mg on Day 1 and at 0.5 mg, 2.0 mg, and 5.0 mg on Day 12; and
   No treatment effect on Pz-O1 or Pz-O2 (eyes open and eyes dosed) at any dose on Day 1 or Day 12;
Delta-power:
   Significant decrease in Fz-Cz (eyes closed) at all dose levels on Day 12;
   Significant decrease in Fz-Cz (eyes open) only at 1.0 mg on Day 12;
   Significant decrease in Pz-O1 (eyes closed) at 1.0 mg, 1.5 mg, 2.0 mg, and 5.0 mg on Day 12;
   Significant decrease in Pz-O2 (eyes closed) at all dose levels on Day 12; and
   Significant decrease in Pz-O1 and Pz-O2 (eyes open) at 1.5 mg, 2.0 mg, and 5.0 mg on Day 12;
Gamma-power:
   Significant increase in Fz-Cz (eyes closed and eyes open) at 2.0 mg on Day 1 and at 2.0 mg and 5.0 mg on Day 12;
   Significant decrease in Pz-O1 (eyes dosed and eyes open) at 1.5 mg, 2.0 mg, and 5.0 mg on Day 1;
   No treatment effect on Pz-O2 (eyes closed) for any dose level except for 5.0 mg on Day 12; and
   No treatment effect on Pz-O2 (eyes open) for any dose level; and
Theta-power:
   Significant decrease in Fz-Cz (eyes closed and eyes open) at 1.0 mg, 1.5 mg, 2.0 mg, and 5.0 mg on Day 12; and
   Significant decrease in Pz-O1 and Pz-O2 (eyes closed and eyes open) at 1.5 mg, 2.0 mg, and 5.0 mg on Day 12.

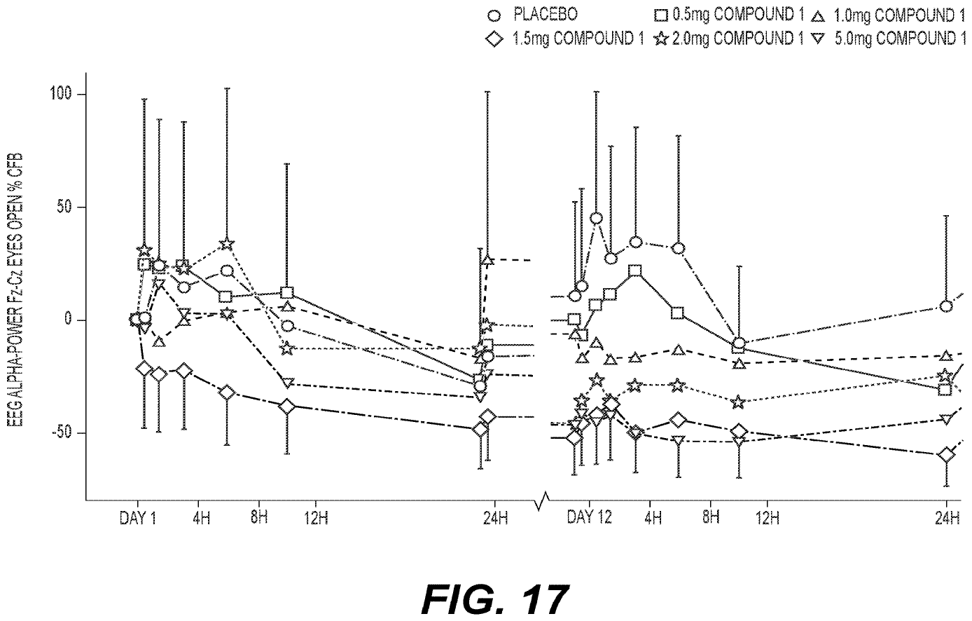

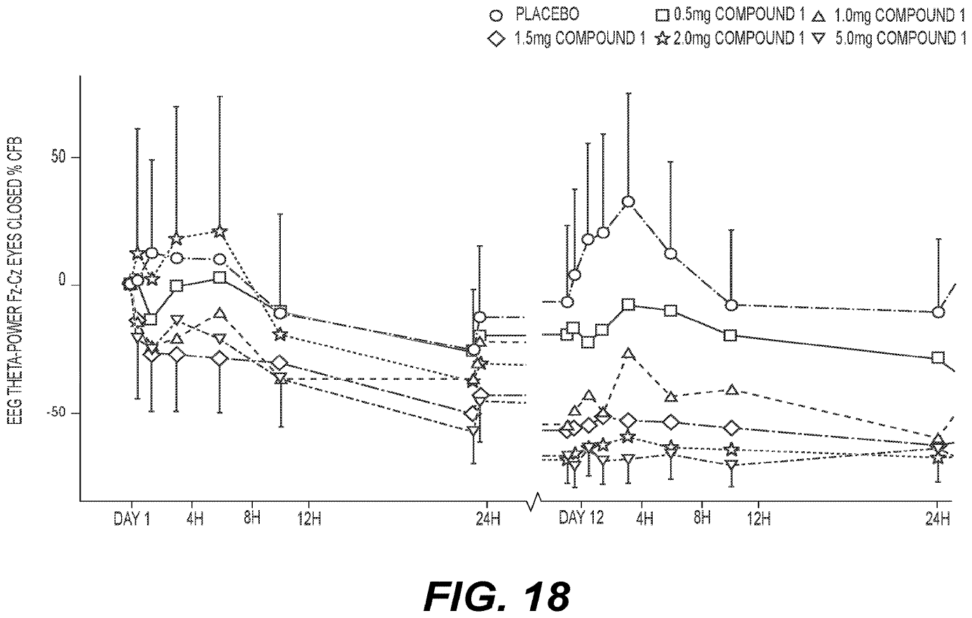

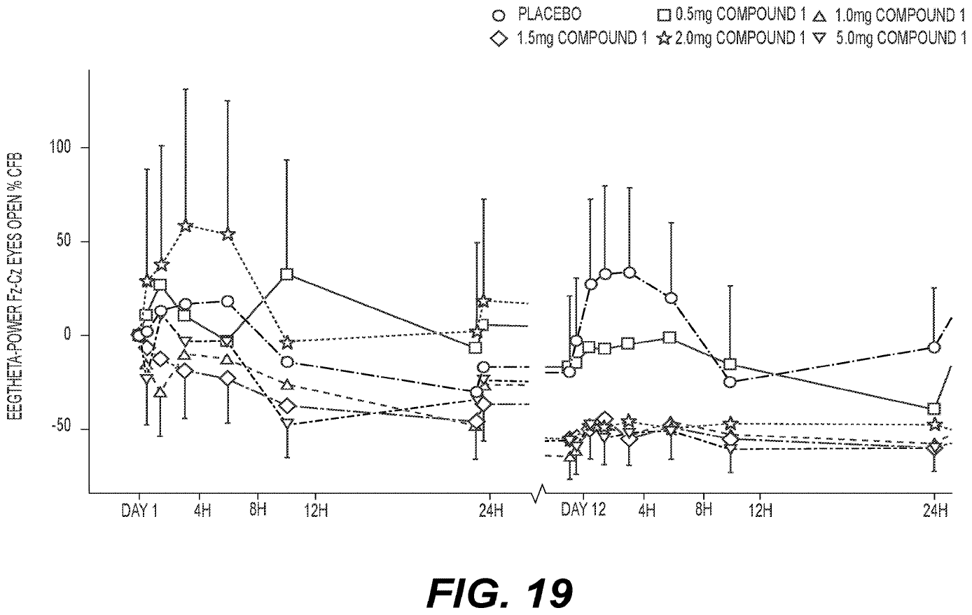

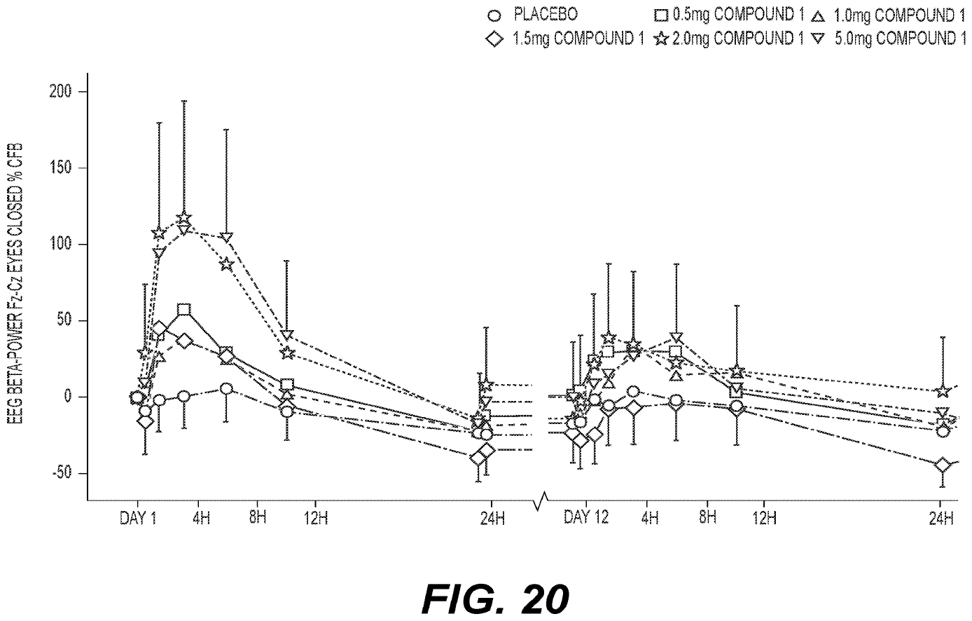

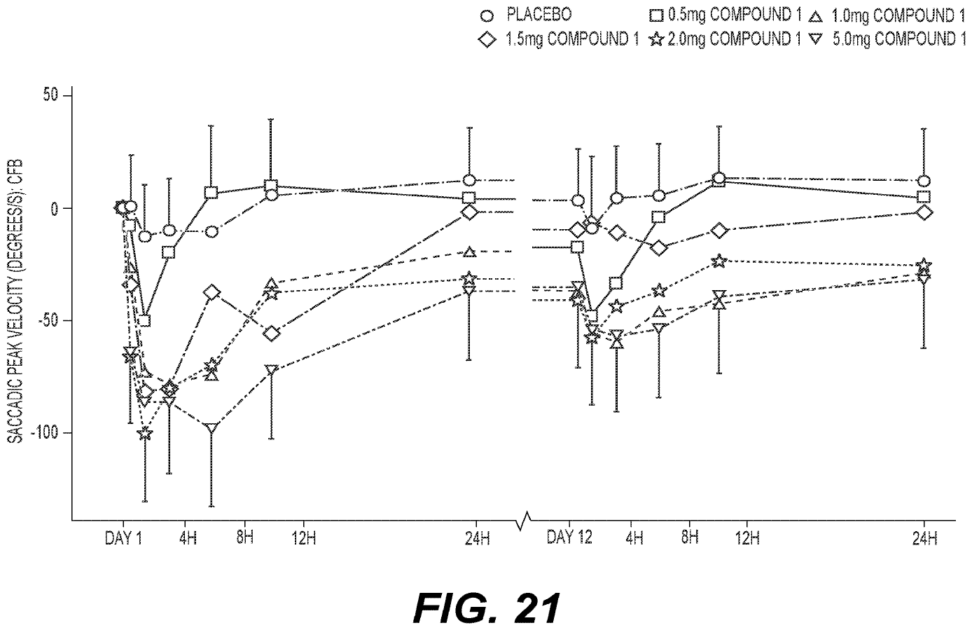

In summary, the most consistent effects on qEEG were decreased alpha- and theta-power, and increased beta-power for Compound 1>1.5 mg on Day 12. However, the effects on alpha- (FIG. 17) and theta-power (FIG. 18 and FIG. 19) on Day 12 were suggestive of dose or concentration-dependence, which did not seem to be the case for beta-power on Day 12 (FIG. 20).

### Summary of Pharmacodynamic Findings

Compound 1 reduced SPV to a comparable extent at 1.0 mg, 2.0 mg, and 5.0 mg on Day 1, an effect that persisted up to Day 12 and supports a sustained effect on arousal during repeated dosing.

Although Compound 1 significantly decreased adaptive tracking at 2:1.0 mg and significantly increased body sway at 5.0 mg on Day 1, that was not the case for either on Day 12, indicating an acute effect on sustained attention that subsided over time and lack of effect on overall psychomotor function and postural balance at doses<5.0 mg during repeated dosing.

Compound 1 significantly decreased VAS alertness at 2.0 mg on Day 1 but not on Day 12, while VAS alertness at 5.0 mg remained unaffected on Day 1 and decreased significantly on Day 12, indicating an inconsistent effect on subjective sedation, which can be considered in line with the adaptive tracking and body sway findings.

Compound 1 did not demonstrate consistent psychotomimetic (VAS Bowdle) or autonomic nervous system (pupil size) effects.

The effects of Compound 1 on the WLT were rather inconsistent. Compound 1 5.0 mg negatively affected memory encoding, retrieval, and consolidation on Day 1 but not at Day 12, which supports tolerance to such effects following repeated dosing.

Compound 1 demonstrated the most consistent effects on qEEG at doses>1.5 mg, with decreased alpha- and theta-power on Day 12 that were suggestive of dose- or concentration-dependence and target engagement, but increased beta-power on Day 12 did not suggest dose- or concentration-dependence.

### Discussion and Overall Conclusions

Compound 1 represents a new therapeutic modality for the potential treatment of epilepsy, anxiety, spasticity, or other CNS disorders. This study was conducted in healthy male and female participants to assess the safety, tolerability, PK, and PD of Compound 1.

Twelve days of consecutive dosing with Compound 1 0.5 mg, 1.0 mg, 1.5 mg, 2.0 mg, and 5.0 mg was generally safe and well tolerated in healthy volunteers. No deaths, SAEs, or TEAEs leading to discontinuation were reported.

The majority of TEAEs were mild in severity. Two TEAEs of moderate somnolence at Compound 1 2.0 mg and 5.0 mg and 1 TEAE of moderate balance disorder at Compound 1 5.0 mg were reported. All TEAEs were transient and required no treatments or interventions. Although somnolence and fatigue were the most frequently reported TEAEs, these also occurred in approximately 50% of placebo-treated participants. There was no clear relationship between increase in dose and incidence of somnolence and fatigue. Dizziness was experienced in 20.0% of participants receiving Compound 1, but that was not the case for any participant receiving placebo; postural dizziness was reported in 30.0% and 10.0% of participants receiving Compound 1 and placebo, respectively.

Compound 1 up to 5.0 mg did not demonstrate a difference from placebo in sedation as measured using the MOAA/S.

Compound 1 was rapidly absorbed, with a median Tₘₐₓ varying from 3 h up to 2.0 mg to 4.5 h at 5.0 mg on Day 1.

Mean peak concentrations (Cₘₐₓ) and mean area-under-the curve 0 to 24 h (AUC₀₋₂₄) generally increased dose- and concentration-proportionally.

Compound 1 concentration followed bi-exponential decline with apparent terminal t_{1/2}±SD ranging between 39±9 h for 5.0 mg and 66±24 h for 0.5 mg.

Steady-state was reached by 6 to 12 days of repeated dosing and PK variability was acceptable.

Unexpectedly, Compound 1 reduced SPV to a comparable extent whether at 1.0 mg, 2.0 mg, or 5.0 mg, and sustained such decreases up to 12 days.

Although Compound 1 significantly decreased adaptive tracking at 21.0 mg and significantly increased body sway at 5.0 mg acutely, that was not the case after repeated dosing up to 12 days.

There was no consistent effect on VAS alertness and there were no indications for psychotomimetic or autonomic nervous system effects acutely or following repeated dosing up to 12 days.

Compound 1 demonstrated no consistent effect on the WLT at 52.0 mg, while 5.0 mg negatively affected memory encoding, retrieval, and consolidation acutely until Day 12.

Compound 1 demonstrated sustained reduced arousal, which was not associated with effects on sustained attention or postural stability, subjective sedation, or memory dysfunction following repeated dosing up to 12 days in the dose range between 1.0 mg and 5.0 mg.

Compound 1 demonstrated the most consistent effects on qEEG at doses>1.5 mg, with decreased alpha- and theta-power on Day 12 that were suggestive of dose- or concentration-dependence, but increased beta-power on Day 12 did not suggest dose- or concentration-dependence.

A randomized, double-blind, placebo-controlled, multiple ascending dose study with oral dosing in healthy volunteers for 12 days was performed. Repeated administration of Compound 1 for 12 days was safe and well tolerated up to 5.0 mg. In addition, Compound 1 demonstrated a PD profile indicative of sustained reduced arousal that lacks untoward sedative, psychomotor, or memory effects between 1.0 mg and 5.0 mg following repeated dosing up to 12 days and is supported by qEEG findings that confirm persistent central target engagement. Observed Compound 1 PK parameters support QD dosing.

1. A method of treating an anxiety disorder in a subject in need thereof, comprising administration of a dosage form to the subject, wherein:
the administration is oral once daily administration;
the dosage form comprises 1, 2, or 3 tablets or capsules comprising 2′,6-difluoro-5′-(3-(2-hydroxypropan-2-yl)imidazo[1,2-b][1,2,4]triazin-7-yl)-[1,1′-biphenyl]-2-carbonitrile or a pharmaceutically acceptable salt thereof;
the dosage form comprises about 0.5 to 5 mg of 2′,6-difluoro-5′-(3-(2-hydroxypropan-2-yl)imidazo[1,2-b][1,2,4]triazin-7-yl)-[1,1′-biphenyl]-2-carbonitrile or a pharmaceutically acceptable salt thereof;
the 2′,6-difluoro-5′-(3-(2-hydroxypropan-2-yl)imidazo[1,2-b][1,2,4]triazin-7-yl)-[1,1′-biphenyl]-2-carbonitrile or a pharmaceutically acceptable salt thereof is present in plasma of the subject at a steady-state when undergoing the administration; and
the once daily administration includes at least 6 consecutive days of administration.

2. A dosage form, including:
1) about 1.5 to about 100 μg of 2′,6-difluoro-5′-(3-(2-hydroxypropan-2-yl)imidazo[1,2-b][1,2,4]triazin-7-yl)-[1,1′-biphenyl]-2-carbonitrile or a pharmaceutically acceptable salt thereof per mL of the dosage form; or
2) a dosage amount of about 0.002 to about 1 mg of 2′,6-difluoro-5′-(3-(2-hydroxypropan-2-yl)imidazo[1,2-b][1,2,4]triazin-7-yl)-[1,1′-biphenyl]-2-carbonitrile or a pharmaceutically acceptable salt thereof per kg of a subject; or
3) about 0.5, 0.63, 1.0, 1.3, 1.5, 1.9, 2.0, 2.5, 5.0, or 6.25 mg of 2′,6-difluoro-5′-(3-(2-hydroxypropan-2-yl)imidazo[1,2-b][1,2,4]triazin-7-yl)-[1,1′-biphenyl]-2-carbonitrile or a pharmaceutically acceptable salt thereof; or
4) about 1, 2, 3, 4, 5, 6, 7, 8, 9, 10, 11, 12, or 13 μmol of 2′,6-difluoro-5′-(3-(2-hydroxypropan-2-yl)imidazo[1,2-b][1,2,4]triazin-7-yl)-[1,1′-biphenyl]-2-carbonitrile or a pharmaceutically acceptable salt thereof.

3. The dosage form of claim 2, wherein the dosage form includes about 20 μg/mL of 2′,6-difluoro-5′-(3-(2-hydroxypropan-2-yl)imidazo[1,2-b][1,2,4]triazin-7-yl)-[1,1′-biphenyl]-2-carbonitrile or a pharmaceutically acceptable salt thereof.

4. The dosage form of claim 2, further including a pharmaceutically acceptable carrier.

5. The dosage form of claim 2, wherein the dosage form includes methyl 2-hydroxyethyl cellulose.

6. The dosage form of claim 2, wherein the dosage form includes about 0.1 to about 3% w/w methyl 2-hydroxyethyl cellulose.

7. The dosage form of claim 2, wherein the dosage form includes about 0.5 to about 2% w/w methyl 2-hydroxyethyl cellulose.

8. The dosage form of claim 2, wherein the dosage amount includes about 0.005 mg/kg, about 0.01 mg/kg, about 0.015 mg/kg, about 0.016 mg/kg, about 0.03 mg/kg, about 0.05 mg/kg, about 0.1 mg/kg, about 0.15 mg/kg, about 0.16 mg/kg, about 0.3 mg/kg, or about 1 mg/kg of 2′,6-difluoro-5′-(3-(2-hydroxypropan-2-yl)imidazo[1,2-b][1,2,4]triazin-7-yl)-[1,1′-biphenyl]-2-carbonitrile or a pharmaceutically acceptable salt thereof.

9. The dosage form of claim 2, comprising about 0.5, 0.63, 2.0, or 2.5 mg of 2′,6-difluoro-5′-(3-(2-hydroxypropan-2-yl)imidazo[1,2-b][1,2,4]triazin-7-yl)-[1,1′-biphenyl]-2-carbonitrile or a pharmaceutically acceptable salt thereof.

10. The dosage form of claim 2, which is a solid dosage form.

11. The dosage form of claim 2, which is a liquid dosage form.

12. The dosage form of claim 2, which is an oral dosage form.

13. The dosage form of claim 2, which is housed in at least one container.

14. A method, comprising administering to a subject in need thereof a therapeutically effective amount of the dosage form of claim 2.

15. The method of claim 14, wherein the subject does not experience a decrease in time to non-REM onset of sleep as compared to a subject receiving a corresponding dose of lorazepam.

16. A method, comprising administering to a subject in need thereof a therapeutically effective amount of 2′,6-difluoro-5′-(3-(2-hydroxypropan-2-yl)imidazo[1,2-b][1,2,4]triazin-7-yl)-[1,1′-biphenyl]-2-carbonitrile or a pharmaceutically acceptable salt thereof,
wherein the subject does not experience a decrease in time to non-REM onset of sleep as compared to a subject receiving a corresponding dose of lorazepam.

17. The method of claim 16, wherein delta, theta, or delta and theta electroencephalogram (EEG) oscillation power from the subject are decreased by about 20% or more during non-REM sleep as compared to a subject receiving a corresponding dose of lorazepam.

18. The method of claim 17, wherein the delta, theta, or delta and theta EEG oscillation power is measured at the frontal, parietal, or frontal and parietal cortex of the subject's brain.

19. The method of claim 18, wherein the subject suffers from a GABA_{A} receptor related disease or a disease or disorder of the central nervous system.

20. A method of treating a GABA_{A} receptor related disease or a disease or disorder of the central nervous system in a subject in need thereof, comprising administering to the subject a therapeutically effective amount of the dosage form of claim 2,
optionally wherein the subject does not experience a decrease in time to non-REM onset of sleep as compared to a subject receiving a corresponding dose of lorazepam.

21. A method of treating a GABA_{A} receptor related disease or a disease or disorder of the central nervous system in a subject in need thereof, comprising administering to the subject a therapeutically effective amount of 2′,6-difluoro-5′-(3-(2-hydroxypropan-2-yl)imidazo[1,2-b][1,2,4]triazin-7-yl)-[1,1′-biphenyl]-2-carbonitrile or a pharmaceutically acceptable salt thereof,
wherein the subject does not experience a decrease in time to non-REM onset of sleep as compared to a subject receiving a corresponding dose of lorazepam.

22. A method, comprising administration of the dosage form of claim 2 to a subject, the method further comprising generation of a pharmacokinetic parameter for 2′,6-difluoro-5′-(3-(2-hydroxypropan-2-yl)imidazo[1,2-b][1,2,4]triazin-7-yl)-[1,1′-biphenyl]-2-carbonitrile or a pharmaceutically acceptable salt thereof in a subject, wherein:
the administration of the dosage form is once per day;
the dosage form comprises about 0.5, 1.0, 1.5, or 2.0 mg of 2′,6-difluoro-5′-(3-(2-hydroxypropan-2-yl)imidazo[1,2-b][1,2,4]triazin-7-yl)-[1,1′-biphenyl]-2-carbonitrile or 0.63, 1.3, 1.9, or 2.5 mg of 2′,6-difluoro-5′-(3-(2-hydroxypropan-2-yl)imidazo[1,2-b][1,2,4]triazin-7-yl)-[1,1′-biphenyl]-2-carbonitrile phosphate; and
the pharmacokinetic parameter is a Tₘₐₓ of about 3 hours or a T_{1/2} of about 60-70 hours, or both.

23. A method, comprising administration of the dosage form of claim 2 to a subject, the method further comprising generation of a pharmacokinetic parameter for 2′,6-difluoro-5′-(3-(2-hydroxypropan-2-yl)imidazo[1,2-b][1,2,4]triazin-7-yl)-[1,1′-biphenyl]-2-carbonitrile or a pharmaceutically acceptable salt thereof in a subject, wherein:
the administration of the dosage form is once per day;
the dosage form comprises about 1, 2, 3, 4, or 5 μmol of 2′,6-difluoro-5′-(3-(2-hydroxypropan-2-yl)imidazo[1,2-b][1,2,4]triazin-7-yl)-[1,1′-biphenyl]-2-carbonitrile or 2′,6-difluoro-5′-(3-(2-hydroxypropan-2-yl)imidazo[1,2-b][1,2,4]triazin-7-yl)-[1,1′-biphenyl]-2-carbonitrile phosphate; and
the pharmacokinetic parameter is a Tₘₐₓ of about 3 hours or a T_{1/2} of about 60-70 hours, or both.

24. A method of treating an anxiety disorder in a subject in need thereof, comprising administration of the dosage form of claim 2 to the subject.

25. The method of claim 24, wherein the administration is oral once daily administration.

26. The method of claim 25, wherein the dosage form comprises 1, 2, or 3 tablets or capsules comprising 2′,6-difluoro-5′-(3-(2-hydroxypropan-2-yl)imidazo[1,2-b][1,2,4]triazin-7-yl)-[1,1′-biphenyl]-2-carbonitrile or a pharmaceutically acceptable salt thereof.

27. The method of claim 19, wherein the dosage form comprises about 1.0, 1.3, 2.0, 2.6, 3.0, 3.1, 3.8, 4.0, 4.1, 5.0, or 5.1 μmol of 2′,6-difluoro-5′-(3-(2-hydroxypropan-2-yl)imidazo[1,2-b][1,2,4]triazin-7-yl)-[1,1′-biphenyl]-2-carbonitrile or 2′,6-difluoro-5′-(3-(2-hydroxypropan-2-yl)imidazo[1,2-b][1,2,4]triazin-7-yl)-[1,1′-biphenyl]-2-carbonitrile mono-phosphate.

28. The method of claim 19, wherein the dosage form comprises about 2 mg of 2′,6-difluoro-5′-(3-(2-hydroxypropan-2-yl)imidazo[1,2-b][1,2,4]triazin-7-yl)-[1,1′-biphenyl]-2-carbonitrile or about 2.5 mg 2′,6-difluoro-5′-(3-(2-hydroxypropan-2-yl)imidazo[1,2-b][1,2,4]triazin-7-yl)-[1,1′-biphenyl]-2-carbonitrile mono-phosphate.

29. The method of claim 28, wherein 2′,6-difluoro-5′-(3-(2-hydroxypropan-2-yl)imidazo[1,2-b][1,2,4]triazin-7-yl)-[1,1′-biphenyl]-2-carbonitrile or a pharmaceutically acceptable salt thereof is present in plasma of the subject at a steady-state when undergoing once daily administration of 2′,6-difluoro-5′-(3-(2-hydroxypropan-2-yl)imidazo[1,2-b][1,2,4]triazin-7-yl)-[1,1′-biphenyl]-2-carbonitrile or a pharmaceutically acceptable salt thereof, and the once daily administration includes at least 6 consecutive days of administration.

30. The method of claim 29, wherein the steady-state in plasma of the subject includes a Cₘₐₓ of 2′,6-difluoro-5′-(3-(2-hydroxypropan-2-yl)imidazo[1,2-b][1,2,4]triazin-7-yl)-[1,1′-biphenyl]-2-carbonitrile or a pharmaceutically acceptable salt thereof of about 10-50 ng/mL.

31. (canceled)
